(19) United States
(12) Patent Application Publication
(10) Pub. No.: US 2023/0027285 A1
(43) Pub. Date: Jan. 26, 2023

(54) SMALL UNANNOTATED, NON-CODING RNAS FOR THE DETECTION OF LIVER CANCER

(71) Applicant: Icahn School of Medicine at Mount Sinai, New York, NY (US)

(72) Inventors: Augusto VILLANUEVA, New York, NY (US); Bojan VLOSIC, New York, NY (US); Johann VON FELDEN, New York, NY (US)

(21) Appl. No.: 17/778,598

(22) PCT Filed: Nov. 20, 2020
(86) PCT No.: PCT/US2020/061447

§ 371 Date: May 20, 2022

Related U.S. Application Data
(60) Provisional application No. 62/938,493, filed on Nov. 21, 2019.

(51) Int. Cl. C12Q 1/6886; G01N 33/574; C12Q 1/70; C12Q 1/6806

(52) U.S. Cl. CPC C12Q 1/6886; G01N 33/57438; C12Q 1/706; C12Q 1/6806; C12Q 2600/178

(57) ABSTRACT

The present disclosure relates to methods for detecting hepatocellular carcinoma (HCC) at an early stage where curative therapies are still an option and survival rates are increased. The methods involve the detection of three, small unannotated non-coding RNAs found in the exosomes of patients with early HCC.

### BACKGROUND

In 2030, more than one million people will die from liver cancer (Villanueva 2019). With a 5-year survival rate of 18%, it is the second most lethal malignancy (Jemal et al. 2017). The majority of hepatocellular carcinoma (HCC), the most frequent form of primary liver cancer, occurs in patients with underlying liver disease, mainly due to infections with hepatitis B (HBV) or C (HCV) virus, alcohol abuse or non-alcoholic fatty liver disease (NAFLD) in the context of diabetes and metabolic syndrome (Llovet et al. 2016). There are no available drugs to prevent or reduce the risk of HCC and only a minority (less than 30%) are diagnosed early enough for a potential cure.

Clinical Practice Guidelines recommend biannual surveillance in patients at high risk of HCC development (Marrero et al. 2018; Galle et al. 2018). The gold standard for HCC surveillance including abdominal ultrasound (US), with or without serum alpha-fetoprotein (AFP), has low sensitivity for the detection of early stage HCC (63%) (Tzartzeva et al. 2018). Additionally, HCC surveillance has a poor implementation rate (less than 20% overall) (Singal et al. 2012). Improvement in the area of early diagnosis is urgently needed.

Extracellular vesicles (EVs), including microvesicles and exosomes, are nanoparticles whose nucleic payload is capable of priming receptor cells to modify key cellular functions (Mathieu et al. 2019). EVs are heterogeneous, both in terms of biogenesis and content (van Niel et al. 2018). While larger EVs such as apoptotic bodies mostly contain fragmented DNA, smaller EVs such as exosomes are enriched in non-coding, regulatory small RNAs (sRNAs) (Jeppesen et al. 2019; Murillo et al. 2019; Sun et al. 2018; Zhang et al. 2015). sRNAs arise from thousands of endogenous genes and are part of the genomic “dark matter” of highly abundant yet largely uncharacterized noncoding RNA, with emerging roles in regulating gene expression via post-transcriptional and translational mechanisms. In cancer, EVs are increasingly recognized as key players in tumor initiation and metastasis, mainly through miRNA, prompting their evaluation as early detection and treatment response biomarkers (Mathieu et al. 2019; Kosaka et al. 2016; Hoshino et al 2015; Skog et al. 2008; Chen et al. 2018; Yang et al. 2017; Jim et al. 2017). However, relatively little attention has been paid to characterizing the general expression landscape of circulating exosomal EV sRNA and their precursors in this context regardless of biotype, especially for those expressed from unannotated genomic regions.

### SUMMARY

The present disclosure addresses the needs in the field by providing methods, compositions and kits for detecting, diagnosing and treating hepatocellular carcinoma (HCC), particularly at early, asymptomatic stages, by detecting and/or quantifying a three-signature small RNA cluster (smRC) in exosomes released from tumor cells. The methods are based on the surprising finding that this three signature smRC (“3-smRC signature”) from exosomes can detect and are diagnostic for HCC, even at early stages, and can be detected in biological fluids obtained by minimally invasive or non-invasive techniques, particularly blood and urine.

Thus, one embodiment of the present disclosure is a method of detecting three small unannotated non-coding RNAs specific for hepatocellular carcinoma in a subject comprising:

a. isolating or purifying exosomes from a sample from the subject;

b. extracting RNA from the exosomes;

c. contacting RNA from the exosomes with at least one primer which is a synthetic nucleic acid and wherein the primer sequence has the nucleotide sequence of SEQ ID NOs: 1 or 2 or a fragment or variant thereof, or the nucleotide sequence complementary to SEQ ID NOs: 1 or 2 or a fragment or variant thereof;

d. further contacting RNA from the exosomes with at least one primer which is a synthetic nucleic acid and wherein the primer sequence has the nucleotide sequence of SEQ ID NOs: 3 or 4 or a fragment or variant thereof or the nucleotide sequence complementary to SEQ ID NOs: 3 or 4 or a fragment or variant thereof;

e. further contacting RNA from the exosomes with at least one primer which is a synthetic nucleic acid and wherein the primer sequence has the nucleotide sequence of SEQ ID NOs: 5 or 6 or a fragment or variant thereof or the nucleotide sequence complementary to SEQ ID NOs: 5 or 6 or a fragment or variant thereof;

f. subjecting the RNA and the primers to amplification conditions; and

g. determining the presence or absence of amplification products, wherein the presence of amplification products indicates the presence of the small unannotated non-coding RNAs specific for hepatocellular carcinoma in the sample.

A further embodiment of the present disclosure is a method of detecting and/or diagnosing hepatocellular carcinoma in a subject comprising:
a. isolating or purifying exosomes from a sample from the subject;
b. extracting RNA from the exosomes;
c. assaying the RNA extracted from the exosomes for the levels of three small unannotated non-coding RNAs, wherein the three, small unannotated non-coding RNAs have the nucleotide sequences SEQ ID NOs: 1, 3, and 5;
d. comparing the levels of the three, small unannotated non-coding RNAs from the sample to reference levels of the three, small unannotated non-coding RNAs; and
e. detecting and/or diagnosing that the subject has hepatocellular carcinoma when the levels of the three, small unannotated non-coding RNAs are increased compared to the reference levels.

Yet a further embodiment of the present disclosure is a method for detecting and/or diagnosing hepatocellular carcinoma in a subject, comprising:
a. isolating or purifying exosomes from a first sample from the subject;
b. extracting RNA from the exosomes;
c. assaying the RNA extracted from the exosomes for the levels of three small unannotated non-coding RNAs, wherein the three, small unannotated non-coding RNAs have the nucleotide sequences SEQ ID NOs: 1, 3 and 5;
d. comparing the levels of the three, small unannotated non-coding RNAs from the sample to reference levels of the three, small unannotated non-coding RNAs;
e. assaying the level of alpha-fetoprotein in a second sample from the subject;
f. comparing the level of alpha-fetoprotein in the second sample from the subject to a reference level of alpha-fetoprotein; and
g. detecting and/or diagnosing that the subject has hepatocellular carcinoma when the levels of the three, small unannotated non-coding RNAs and the alpha-fetoprotein are increased compared to the reference levels.

In some embodiments, the first sample and the second sample are the same. In some embodiments, the first sample and the second sample are different.

In some embodiments, a formula is used to aid in the detection and or diagnosis of HCC in the subject.

Thus, a further embodiment of the present disclosure is a method of detecting and/or diagnosing hepatocellular carcinoma in a subject comprising:
a. isolating or purifying exosomes from a sample from the subject;
b. extracting RNA from the exosomes;
c. assaying the RNA extracted from the exosomes for the levels of three small unannotated non-coding RNAs, wherein the three, small unannotated non-coding RNAs have the nucleotide sequences SEQ ID NOs: 1, 3 and 5 and are denoted smRC 119591, smRC 135709 and smRC 48615;
d. calculating the risk of hepatocellular carcinoma using the levels of the small unannotated non-coding RNAs obtained in step c. in the formula:

HCC probability˜(1+exp(−coef))\hat{ }(−1), wherein,
coef=epsilon+alpha*smrc_119591+beta*smrc_135709+gamma*smrc_48615, and
wherein alpha=[1.5, 1.9]; beta=[1.5, 1.9]; gamma=[5, 1.2]; and epsilon=[1.2, 0.8]; and
e. detecting and/or diagnosing the subject has hepatocellular carcinoma when a subject has an overexpression of the three, small unannotated non-coding RNAs according to the formula.

The risk of hepatocellular carcinoma in the patient can be determined by the formula set forth above, which will return the probability risk that the patient has HCC. The value of the HCC probability risk obtained from this equation ranges from 0 to 1. Zero means 0% probability of having HCC, and one means 100% probability of having HCC.

In some embodiments, the detecting and/or diagnosing that the subject has hepatocellular carcinoma includes comparing the HCC probability to a threshold value and wherein when the HCC probability exceeds the threshold value, automatically detecting and/or diagnosing the patient as having hepatocellular carcinoma.

In some embodiments, for maximum sensitivity to early disease, the threshold probability is greater than or equal to 40%. In some embodiments, for maximum specificity to early HCC specifically, the threshold probability is greater than or equal to 60%.

In some embodiments, the methods further comprise detecting alpha-fetoprotein in a second sample from the subject. In some embodiments, the first sample and the second sample are the same. In some embodiments, the first sample and the second sample are different.

A further embodiment is a method for treating hepatocellular carcinoma in a subject, comprising:
a. purifying exosomes from a sample from the subject;
b. extracting RNA from the exosomes;
c. assaying the RNA extracted from the exosomes for three, small unannotated non-coding RNAs, wherein the three, small unannotated non-coding RNAs have the nucleotide sequences SEQ ID NOs: 1, 3 and 5;
d. comparing the levels of the three, small unannotated non-coding RNAs from the sample to reference levels of the three, small unannotated non-coding RNAs;
e. detecting and/or diagnosing that the subject has hepatocellular carcinoma when the levels of the three, small unannotated non-coding RNAs are increased compared to the reference levels; and
f. treating the subject, wherein the treatment of the subject includes treatments for the early stages of hepatocellular carcinoma, including but not limited to surgical therapies and tumor ablation and immune based therapies.

Yet a further embodiment of the present disclosure is a method of treating hepatocellular carcinoma in a subject comprising:
a. isolating or purifying exosomes from a sample from the subject;
b. extracting RNA from the exosomes;
c. assaying the RNA extracted from the exosomes for the levels of three, small unannotated non-coding RNAs, wherein the three, small unannotated non-coding RNAs have the nucleotide sequences SEQ ID NOs: 1, 3 and 5 and are denoted smRC 119591, smRC 135709 and smRC 48615;
d. calculating the risk of hepatocellular carcinoma using the levels of the small unannotated non-coding RNAs obtained in step c. in the formula:

HCC probability˜(1+exp(−coef)\hat{ }(−1), wherein,
coef=epsilon+alpha*smrc_119591+beta*smrc_135709+gamma*smrc_48615, and
wherein alpha=[1.5, 1.9]; −beta=[1.5, 1.9]; gamma=[5, 1.2]; and epsilon=[1.2, 0.8];
e. detecting and/or diagnosing the subject has hepatocellular carcinoma when a subject has an overexpression of the three, small unannotated non-coding RNAs according to the formula; and
f. treating the subject, wherein the treatment of the subject includes treatments for the early stages of hepatocellular carcinoma, including but not limited to surgical therapies and tumor ablation and immune based therapies.

The risk of hepatocellular carcinoma in the patient can be determined by the formula set forth above, which will return the probability risk that the patient has HCC. The value of the HCC probability risk obtained from this equation ranges from 0 to 1. Zero means 0% probability of having HCC, and one means 100% probability of having HCC.

In some embodiments, the detecting and/or diagnosing that the subject has hepatocellular carcinoma includes comparing the HCC probability to a threshold value and wherein when the HCC probability exceeds the threshold value, automatically detecting and/or diagnosing the patient as having hepatocellular carcinoma.

In some embodiments, for maximum sensitivity to early disease, the threshold probability is greater than or equal to 40%. In some embodiments, for maximum specificity to early HCC specifically, the threshold probability is greater than or equal to 60%.

In some embodiments, the methods of treatment further comprise detecting alpha-fetoprotein in a second sample from the subject. In some embodiments, the first sample and the second sample are the same. In some embodiments, the first sample and the second sample are different.

In some embodiments of all of the foregoing methods, the methods further comprise a confirmatory detection and/or diagnosis of HCC in the subject including further tests or procedures including but not limited to an ultrasound, the detection of alpha-fetoprotein, magnetic resonance imaging (MRI), computed tomography (CT), biopsy or combinations thereof.

In some embodiments of all of the foregoing methods, the subject is at risk for HCC. In some embodiments, the subject has HBV infection, HCV infection, HCV infection with advanced fibrosis, cirrhosis of any cause, NAFLD, or combinations thereof. In some embodiments, the subject has a history of HCC. In some embodiments, the subject is being treated for HCC and the method can be used to determine the effectiveness of the treatment.

In some embodiments of all of the foregoing methods, the sample may be any sample that includes exosomes suitable for detection, purification or isolation. Sources of samples include blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes. Preferred samples include blood, serum, plasma, and urine.

The exosomes may be purified from the sample using ultracentrifugation. In some embodiments, the purification of the exosomes enriches for small EVs (median size of 120 nm).

Provided for herein are synthetic nucleic acids, including probes, primers and primer sets, for practicing any of the methods disclosed herein.

Also provided for are kits for practicing any of the methods disclosed herein.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1—Summary and quality assessment of EV separation process from human plasma samples. FIG. 1A shows a schematic view of study flow diagram with different cohorts, and available specimen and separation analysis of each cohort. FIG. 1B is a representative transmission electron microscopy image of prostate cancer serum isolate. FIG. 1C is a representative transmission electron microscopy image of HCC plasma isolate. FIG. 1D are graphs of nanoparticle tracking analysis results in the plasma isolate of a control (left panel) and HCC (right panel) patient with corresponding size distribution and estimated particle concentration. FIG. 1E is a representative western blotting image of protein lysate from isolate against TSG101 (approximately 55 kDa) in two controls (left) and two HCC (right) patients. FIG. 1F is images of immunoblotting of the isolates with Exoview™. Isolates were captured by indicated antibodies (CD81, CD63, CD9, control IgG) on a chip and stained with CD9 antibodies to visualize different EV subpopulations in one control and three HCC samples (#1.a and #1.b represent technical replicates from the same patient). FIG. 1G is images of immunolabeling of the isolates with Exoview™. Isolates were captured by indicated antibodies (CD81, CD63, CD9, control IgG) on a chip and stained with CD81 antibodies to visualize different EV subpopulations in one control and three HCC samples (#1.a and #1.b represent technical replicates from the same patient). FIG. 1H is a heatmap of the correlations of estimated cargo profiles with the exRNA expression profiles. exRNA expression in units of normalized expression is correlated with key RNA species distinguishing the 6 cargo types (CTs, columns) previously identified. CT4 is heavily enriched, i.e., ncRNA profiles 58-75 are heavily enriched, indicating highly EV specific origin of exRNA.

FIG. 2—Key properties of small RNA clusters (smRCs). FIG. 2A shows the minimum coverage and sub-read length minimal spacing that define smRCs. Read tiling complexity captures heterogeneity of smRC read distribution. FIG. 2B is a density plot of smRC length. FIG. 2C shows the correlation of smRC expression across different EV extraction methods (i.e., ultracentrifugation (UC) versus nanoDLD). FIG. 2D shows the correlation of smRC expression across different biofluids (i.e., serum versus urine) using UC. FIG. 2E is a graph of the percentage of smRC captured by both UC and nanoDLD EV isolation. FIG. 2F is a chart of the distribution of percentage overlap of smRCs onto all known hg38 RNA biotypes. Low overlap (<<1) indicates smRC does not contain whole RNA biotype, high or total overlap (approximately 1) indicates RNA biotype contained within smRC. FIG. 2G are plots of given biotype abundance percentage (among all RNA biotypes in hg38 annotation) versus smRC overlap percentage as above. Abundance percentage quantifies the frequency of a given RNA biotype among all others. FIG. 2H are plots that are the same as FIG. 2G except the curves are derived from a random genomic distribution matching number and size of smRC. FIG. 2I is a volcano plot for differential expression between smRC of cellular versus EV origin. FIG. 2J is a graph of the maximum value logFC among all significant smRCs as a function of the length of the smRCs peak consensus sequence. FIG. 2K shows the smRC complexity as a function of peak coverage colored by differential smRC expression between cellular and exRNA origin. smRCs enriched in exRNAs present with low complexity and higher peak coverage, whereas cellular smRCs are more frequently of high complexity and lower peak coverage. FIG. 2L shows the correlation of a single smRC expression between RNAseq and RT-PCR in the prostate cancer cohort.

FIG. 3—smRC in HCC biomarker discovery cohort. FIG. 3A is a plot of a principal component analysis (PCA) for HCC biomarker discovery cohort. FIG. 3B shows expression of each smRC between chronic liver disease controls (CLD, n=5), HCC patients (n=10), and patients with other non-HCC malignancies (n=142) (HCC biomarker discovery cohort, RNAseq data). FIG. 3C is a graph of the correlation EV smRC 119591 expression between RNAseq and RT-PCR in the HCC discovery cohort. FIG. 3D is a graph of the correlation EV smRC 135709 expression between RNAseq and RT-PCR in the HCC discovery cohort. FIG. 3E is a graph of the correlation EV smRC 48615 expression between RNAseq and RT-PCR in the HCC discovery cohort.

FIG. 4—Performance of 3-smRC signature in a phase 2 biomarker study. FIG. 4A is a plot of the expression for each smRC between HCC patients and chronic liver disease controls (CLD) (center line, median: box limits, upper and lower quartiles; whiskers, 1.5× interquartile range; points, outliers) (RT-PCR data). FIG. 4B shows the expression for each smRC between HCC patients (n=105), chronic liver disease controls (CLD, n=85), and patients without chronic liver disease (noCLD, n=19) (RT-PCR data). FIG. 4C shows the quantification of the 3-smRC early detection signature in 42 patients, including EV isolation from plasma, RNA extraction and RT-qPCR. These two independent experiments yielded a correlation coefficient of 0.83 (p<0.001). FIG. 4D shows the longitudinal analysis of smRC expression in 30 patients with available sequential blood samples before and after HCC treatments (responders n=13, early recurrence, n=17, paired t-test). Displayed is the smRC expression as delta between the ct values of the spike-in control and respective smRC. Smaller delta equals higher expression of the smRC. FIG. 4E shows the expression of smRC48615 EV-depleted versus EV enriched (RT-PCR data).

FIG. 5—smRC in ‘HCC biomarker validation’ cohort. FIG. 5A is a calibration curve for penalized smRC logistic regression model to predict early HCC, with mean error 0.04. FIG. 5B shows a nomogram for 3-smRC signature to predict early stage HCC.

FIG. 6—Assay robustness and smRC dynamics. FIG. 6A is a ROC curve for maximized gain-of-certainty across repeated cross validation. Each point represents a pair of sensitivities and specificities that maximize gain-in-certainty (i.e., sensitivity+specificity) from a test validation ROC curve, whose AUC colors the point. The loess curves trace the best density fit of points across this space, with 95% confidence intervals shown in gray. FIG. 6B shows the AFP and smRC correlation plot. FIG. 6C shows the bootstrap validation parameters for smRC and smRC+AFP model, respectively. Dxy: Somers' rank correlation between the observed HCC status and predicted HCC probabilities; Emax: maximum absolute calibration error on probability scale; B: Brier score; g: Gini's mean difference of log-odds between HCC and CLD; gp: Gini's mean difference in probability scale; AUC: Area Under the Receiver Operating Curve.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “hepatocellular carcinoma (HCC)” means a primary malignancy of the liver and occurs predominantly in patients with underlying chronic liver disease and cirrhosis. The cell(s) of origin are believed to be the hepatocytes, although this remains the subject of investigation. Tumors progress with local expansion, intrahepatic spread, and distant metastases.

As used herein, the term “subject” or “patient” as used herein refers to a mammal, preferably a human, for whom treatment can be provided.

As used herein, the term “exosomes,” refers to a membranous particle having a diameter (or largest dimension where the particles is not spheroid) of between about 10 nm to about 5000 nm, more typically between 30 nm and 1000 nm, and most typically between about 50 nm and 200 nm, wherein at least part of the membrane of the exosomes is directly obtained from a cell membrane. Most commonly, exosomes will have a size (average diameter) that is up to 5% of the size of the donor cell. Therefore, especially contemplated exosomes include those that are shed from a cell. Platelets or their secreted particles are specifically excluded from this definition of exosomes.

As used herein, the term “sample” refers to any sample suitable for the methods provided by the present embodiments. The sample may be any sample that includes exosomes suitable for detection or isolation. Sources of samples include blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes. In one aspect, the sample is a blood sample, including, for example, whole blood or any fraction or component thereof including serum and plasma. A blood sample suitable for use with the present disclosure may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

The terms “reference value” or “reference levels” as used herein can mean an amount or a quantity of a particular protein or nucleic acid in a sample. The sample can be from a subject not suffering from hepatocellular carcinoma but at high risk for liver cancer. The sample can be from a healthy subject, not suffering from disease. A reference value or level can be a known value or level of a particular protein or nucleic acid, such as one in publications. A reference value or level may also mean an amount or a quantity of a particular protein or nucleic acid in a sample from a patient at another time point in the disease and/or treatment.

The terms “treat”, “treatment”, and the like refer to a means to slow down, relieve, ameliorate or alleviate at least one of the symptoms of the disease, or reverse the disease after its onset.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Abbreviations

HCC—hepatocellular carcinoma
smRC—small RNA cluster
EV—extracellular vesicles
AFP—alpha-fetoprotein
HBV—hepatitis B
HCV—hepatitis C
NAFLD—non-alcoholic fatty liver disease
UC—ultracentrifugation

Shown herein is a novel solution to a key barrier in the field of exRNA derived cancer biomarkers. This study departed from previous exRNA characterization studies, which are restricted to quantifying expression of known (i.e., annotated) transcripts (Mjelle et al. 2019; Sun et al. 2020). Thus, the substantial component of unannotated exRNA were not discarded nor was there a simple focus on a particular RNA biotype (e.g., miRNA) (Lee et al. 2019). Instead, a novel, scalable, and data-driven view of the entire small exRNA landscape unfettered by incomplete and emerging prior knowledge was used. This approach allowed the identification and validation of novel circulating biomarkers for the detection of curable, early stage HCC.

By de novo characterizing the unknown non-coding small exRNA landscape across EV isolation technologies, biofluid, and cancer type, the key properties of exRNA associated smRCs, including their clinical application in early cancer detection were defined. These properties indicated that the tractable smRC-based quantification of novel, unannotated, small RNA expression signatures is feasible across different EV isolation techniques applied to different biofluids, potentially offering a completely novel, data-driven strategy for increasing the sensitivity of EV-driven biomarker discovery. It is important to emphasize that multiple small functional noncoding RNA can arise from transcriptional post-processing of a single larger RNA precursor gene, so smRCs estimate the overlooked underlying expression profile of small RNA precursor genes and thereby facilitate accurate quantification, differential expression, and motif discovery of unknown, heterogeneous, small RNA dominated exRNA payloads. In this sense, smRCs might more accurately measure the information content of exRNA.

Applying this approach to a separate HCC plasma-based exRNA dataset, a 3-smRC (unannotated), HCC specific signature, was derived which was then validated in an independent HCC cohort (‘HCC biomarker validation cohort’, n=209) to discriminate patients with incipient HCC from controls at high-risk of cancer.

Importantly, the exRNA-derived smRC signature was developed as a method for early HCC detection in the context of cancer surveillance which directly determined the patient population deliberately selected for this study, as extensively outlined in clinical guidelines (Marrero et al. 2018; EASL 2018). Briefly, these guidelines explicitly underscore the urgent clinical need for new tools to detect patients with early stage HCC, as they can be cured if diagnosed at this stage. The HCC specificity of the 3 smRC signature was confirmed in a dataset of 142 patients with other malignancies. The study herein purposely chose to test the early detection biomarker candidates in the context of the hardest possible scenario of distinguishing between chronic liver disease and very early, curable, HCC. The signature was independently validated in more than 200 patients, where it was demonstrated of its ability to accurately detect patients with early stage HCC. It was demonstrated that the 3-smRC signature not only outperforms the recommended surveillance tools (serum alphafetoprotein (AFP) combined with abdominal ultrasound) (Tzartzeva et al. 2018) but is complementary to AFP and in combination further maximizes HCC detection rates. There are other approaches currently under evaluation for early HCC detection using other liquid biopsy analytes, mostly involving circulating DNA (Felden et al. 2020). Blood-based DNA mutation and methylation studies have shown comparable performance to the 3-smRC signature disclosed herein (Xu et al. 2017; Smith et al. 2014; Qu et al. 2019). The main difference with this study is that it is exclusively an early-stage cohort and not HCC patients at more advanced stages.

Thus, disclosed herein is a blood-based, minimally-invasive, operator-independent surveillance test to detect HCC at early stages that overcomes the limitations in HCC surveillance (i. e. low implementation rate and suboptimal performance of surveillance tools).

The 3-smRC signature yielded an Area under the Receiver Operating Curve (AUC) of 0.87, 86% sensitivity, 91% specificity, and a positive predictive value of 89%.

A composite approach with the 3-smRC signature plus AFP yielded an Area under the Receiver Operating Curve (AUC) of 0.93, lower Brier score of 0.11, and better test performance (85% sensitivity, 94% specificity, and positive predictive value of 95%).

3-smRC Signature (Three Small Unannotated Non-Coding RNAs) for Early Detection of Hepatocellular Carcinoma (HCC)

The present disclosure provides for a 3-smRC signature or three small unannotated non-coding RNAs extracted from exosomes isolated from samples from patients, which are indicative and predictive of hepatocellular carcinoma.

The small nonannotated non-coding RNAs included in the 3-smRC signature are identified as follows:

smRC 119591 is located in the unannotated region of chromosome 8 (chr8:137627017-137627182). The peak consensus sequence, i.e., the nucleic acid sequence of smRC 119591, is CCUCUUCUUAACACC (SEQ ID NO: 1). The target sequence used for PCR to obtain levels of smRC 119591 in a sample is UUGUCCUCUUCUUAACACC (SEQ ID NO: 2).

smRC 135709 is located in the unannotated region of chromosome 10 (chr10:70817194-70818087). The peak consensus sequence, i.e., the nucleic acid sequence of smRC 135709, is CCUUCCCGUACUACC (SEQ ID NO: 3). The target sequence used for PCR to obtain levels of smRC 135709 in a sample is CUCCCUUCCCGUACUACC (SEQ ID NO: 4).

smRC 48615 is located in the unannotated region of chromosome 3 (chr3:103950043-103953627). The peak consensus sequence, i.e., the nucleic acid sequence, is CUCUUUACAGUGACC (SEQ ID NO: 5). The target sequence used for PCR to obtain levels of smRC 135709 in a sample is UGUCUCUUUACAGUGACC (SEQ ID NO: 6).

See Table 2.

These three, small unannotated non-coding RNAs are useful for the detection and/or diagnosis of hepatocellular carcinoma accurately, especially at an earlier stage than is now possible, allowing for better treatment options, i.e., curative therapies, and better survival rates.

In certain aspects, the disclosure is directed to an isolated nucleic acid sequence as provided in any of SEQ ID NOs: 1-6.

In certain aspects, the disclosure is directed to an isolated nucleic acid complementary to any of SEQ ID NOs: 1-6.

Polynucleotides homologous to the sequences illustrated in SEQ ID NOs: 1-6, can be identified, e.g., by hybridization to each other under stringent or under highly stringent conditions. The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. The stringency of a hybridization reflects the degree of sequence identity of the nucleic acids involved, such that the higher the stringency, the more similar are the two polynucleotide strands. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). Hybridization conditions for various stringencies are known in the art and are disclosed in detail in at least Sambrook et al.

In certain aspects, the disclosure relates to a synthetic nucleic acid comprising the nucleotides of an isolated (or non-isolated) nucleic acid having the sequence of any of SEQ ID NOs: 1-6; an isolated (or non-isolated) nucleic acid complementary to the sequence of any of SEQ ID NOs: 1-6; an isolated (or non-isolated) nucleic acid having at least about 60% sequence identity to any of SEQ ID NOs: 1-6; an isolated (or non-isolated) nucleic acid having at least about 60% sequence identity to a nucleic acid complementary to the sequence of any of SEQ ID NOs: 1-6; an isolated (or non-isolated) nucleic acid which comprises at least 10 consecutive nucleotides of any of SEQ ID NOs: 1-6; an isolated (or non-isolated) nucleic acid which comprises at least 10 consecutive nucleotides of a nucleic acid complementary to the sequence of any of SEQ ID NOs: 1-6; an isolated (or non-isolated) nucleic acid which comprises at least 10 consecutive nucleotides of a sequence having at least about 60% identity to any of SEQ ID NOs: 1-6; or an isolated (or non-isolated) nucleic acid which comprises at least 10 consecutive nucleotides of a sequence having at least about 60% identity to a nucleic acid complementary to the sequence of any of SEQ ID NOs: 1-6.

In other aspects the disclosure is directed to isolated nucleic acid sequences such as primers and probes, comprising nucleic acid sequences of any of SEQ ID NOs: 1-6 or a nucleic acid complementary to the sequence of any of SEQ ID NOs: 1-6. Such primers and/or probes may be useful for detecting the presence of the small unannotated non-coding RNAs, for example in samples such as blood from a subject, and thus may be useful in the diagnosis of HCC. Such probes can detect RNAs of any of SEQ ID NOs: 1-6 in samples which comprise the small unannotated non-coding RNAs of SEQ ID NOs: 1-6. The isolated nucleic acids which can be used as primer and probes are of sufficient length to allow hybridization with, i.e. formation of duplex with a corresponding target nucleic acid sequence, a nucleic acid sequences of any of SEQ ID NOs: 1-6, or a fragment or variant thereof.

The disclosure is also directed to primer and/or probes which can be labeled by any suitable molecule and/or label known in the art, for example but not limited to fluorescent tags suitable for use in Real Time PCR amplification, for example TaqMan, cybergreen, TAMRA and/or FAM probes; radiolabels, and so forth. In certain embodiments, the oligonucleotide primers and/or probe further comprises a detectable non-isotopic label selected from the group consisting of a fluorescent molecule, a chemiluminescent molecule, an enzyme, a cofactor, an enzyme substrate, and a hapten.

In another aspect, the disclosure provides an oligonucleotide probe, wherein the oligonucleotide probe hybridizes to the nucleic acid target region under moderate to highly stringent conditions to form a detectable nucleic acid target:oligonucleotide probe duplex. In one embodiment, the oligonucleotide probe is at least about 95.5%, about 96%, about 96.5%, about 97%, about 97.5%, about 98%, about 98.5%, about 99%, about 99.5% or about 99.9% complementary to SEQ ID NOs: 1-6.

In certain aspects, the disclosure is directed to primer sets comprising isolated nucleic acids as described herein, which primer sets are suitable for amplification of nucleic acids from samples which comprises the three, small unannotated non-coding RNAs represented by any one of SEQ ID NOs: 1-6, or variants thereof. Primer sets can comprise any suitable combination of primers which would allow amplification of a target nucleic acid sequences in a sample which comprises the three, small unannotated non-coding RNAs represented by any of SEQ ID NOs: 1-6, or variants thereof. Amplification can be performed by any suitable method known in the art, for example but not limited to PCR, RT-PCR, and transcription mediated amplification (TMA).

In certain aspects, the disclosure relates to a primer set for detecting the presence of the three, small unannotated non-coding RNAs and detecting and/or diagnosing HCC in a sample, wherein the primer set comprises at least one synthetic nucleic acid sequence selected from the group consisting of the synthetic nucleic acids described herein.

Primers, primer sets, and probes can be designed by those of skill in the art using the sequences of SEQ ID NOs: 1-6.

Methods of Detection of the Three Small Unannotated Non-Coding RNAs and Detection and/or Diagnosis of Hepatocellular Carcinoma (HCC)

As shown herein, three small unannotated non-coding RNAs extracted from exosomes of samples from subjects are associated with, and can detect and/or diagnose HCC, at early stages. The advantages of the use of these three, small unannotated non-coding RNAs is that they can be detected using non-invasive techniques and can detect and diagnose HCC at very early stages, allowing better treatment options, i.e., curative therapies, and better survival rates.

Thus the present disclosure provides for methods of detecting the levels of the three, small unannotated non-coding RNAs described herein in a sample from a subject and using the levels of the RNAs to detect and/or diagnose HCC in a subject who is at known risk for HCC and/or has cirrhosis of any cause, HBV, HCV, NAFLD or combinations thereof.

The sample can be from any source that would include exosomes suitable for detection and include but are not limited to blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes. Preferred samples for use in the methods are urine, blood and serum.

As disclosed herein the three, small unannotated non-coding RNAs for use in the methods of detection, diagnosis and treatment are contained in extracellular vesicles or exosomes. Thus, a step of isolating and/or purifying the exosomes from the sample is necessary.

One method for exosome isolation and/or purification is ultracentrifugation. Exemplified herein is ultracentrifugation at 120,000 g for about 2 hours as well as ultracentrifugation at 110,000 g for 2 hours two times.

A further method for exosome isolation and/or purification is the so-called nanoDLD technology, which is a size-based EV separation technology that integrates 1024 nanoscale deterministic lateral displacement (DLD) arrays on a single chip capable of parallel processing sample fluids. See Wunsch et al. 2016.

Another method for EV isolation and/or purification is the use of VN96 peptide which binds to canonical heat shock proteins which are found on the exterior of exosomes and EVs, particularly from cells that are under stress such as cancer cells. This binding leads to the exosomes being easily precipitated into a pellet with a brief series of spins in a normal benchtop centrifuge. See Ghosh et al. 2014 and U.S. Pat. No. 8,956,878.

Yet a further method involves the use of a polymer which precipitates the exosomes from the sample.

Several other methods are known in the art and are available commercially.

After the exosomes are purified and/or isolated from the sample, the RNA must be extracted from the exosomes. This can be done by methods known in the art.

After the exosomes are purified and/or isolated from the sample and the RNA extracted from the exosome, the levels of the three, small unannotated non-coding RNAs can be detected using any method known in the art including those that use the primers and probes disclosed herein and not limited to: Southern blots; Northern blots; dot blots; primer extension; nuclease protection; subtractive hybridization and isolation of non-duplexed molecules using, for example, hydroxyapatite; solution hybridization; filter hybridization; amplification techniques such as RT-PCR and other PCR-related techniques such as PCR with melting curve analysis, and PCR with mass spectrometry; fingerprinting, such as with restriction endonucleases; and the use of structure specific endonucleases. mRNA expression can also be analyzed using mass spectrometry techniques (e.g., MALDI or SELDI), liquid chromatography, and capillary gel electrophoresis. Any additional method known in the art can be used to detect the presence or absence of the small unannotated non-coding RNAs.

For a general description of these techniques, see also Sambrook et al. 1989; Kriegler 1990; and Ausebel et al. 1990.

A preferred method of detecting the three, small unannotated non-coding RNAs is polymerase chain reaction (PCR).

As disclosed herein the levels of the three, small unannotated non-coding RNAs can be compared to a reference level or value. In some embodiments, the reference level or value is from a subject not suffering from HCC or liver disease, i.e., a healthy subject. In some embodiments, the reference level or value is from a subject not suffering from HCC but at high risk for liver cancer. In some embodiments, the reference value is from the subject themselves at another time point in the disease or treatment.

In some embodiments, a formula is used to calculate the risk of HCC. The formula is as follows:

HCC probability˜(1+exp(−coef))\hat{ }(−1), wherein,

coef=epsilon+alpha*smrc_119591+beta*smrc_135709+gamma*smrc_48615

where alpha=[1.5, 1.9]; beta=[1.5, 1.9]; gamma=[5, 1.2]; and epsilon=[1.2, 0.8].

The risk of hepatocellular carcinoma in the patient can be determined by the formula, which will return the probability risk that the patient has HCC. The value of the HCC probability risk obtained from this equation ranges from 0 to 1. Zero means 0% probability of having HCC, and one means 100% probability of having HCC.

In some embodiments, the detecting and/or diagnosing that the subject has hepatocellular carcinoma includes comparing the HCC probability to a threshold value and wherein when the HCC probability exceeds the threshold value, automatically detecting and/or diagnosing the patient as having hepatocellular carcinoma.

In some embodiments, for maximum sensitivity to early disease, the threshold probability is greater than or equal to 40%. In some embodiments, for maximum specificity to early HCC specifically, the threshold probability is greater than or equal to 60%.

As shown herein, the disclosed methods of detecting and/or diagnosing HCC using the levels of the three, small unannotated non-coding RNAs is 82% sensitive and 90% specific, with a positive predictive value of 89%. When combined with the detection of alpha-fetoprotein (AFP) the sensitivity is 85%, the specificity is 100% and the positive predictive value is 95%.

Thus, a further embodiment of the present disclosure is a method of detecting and diagnosing HCC by detecting the levels of three, small unannotated non-coding RNAs and further detecting the level of AFP in a sample.

Samples for the detection of AFP again can be from any source that would include protein suitable for detection and include but are not limited to blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes. Preferred samples for use in the methods are urine, blood and serum.

While any method known in the art can be used, preferred methods for detecting and measuring increase levels of the proteins in a protein sample include flow cytometry, quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

The presence or amount of the AFP can be compared to a reference level or value. In some embodiments, the reference level or value is from a subject not suffering from hepatocellular carcinoma or liver cancer. i.e., a healthy subject. In some embodiments, it is a reference level or value known in the art. In some embodiments, the reference level or value is from the subject themselves at another time point in the disease or treatment.

### Methods of Treatment of Hepatocellular Carcinoma (HCC)

The benchmark classification system for HCC classifies patients into five stages of the disease and provides treatment recommendations for each stage. Patients diagnosed at the early stages of HCC, stage 0 and A, have higher survival rates and more treatment options. Treatments for HCC include but are not limited to surgical therapies, tumor ablation, transarterial therapies, and systemic therapies.

Surgical therapies include resection. Resection is recommended for patients with very early stage HCC (BCLC stage 0 or A) and preserved liver function. Patients who are treated by resection have a survival rate of above 60% at five years.

Another surgical therapy is liver transplantation. Patients who are candidates for transplantation again are the early stages of HCC (BCLC stage 0 or A). Additionally, patients should meet the Milan criteria for liver transplantation and have not macrovascular tumor invasion or have extrahepatic spread. Transplantation in these patients is associated with a survival of about 60% to 80% at 5 years and 50% at 10 years. Transplantation has the advantage of not only removing a tumor but curing liver disease.

Tumor ablation is also recommended for patients at the early stages of HCC (BCLC stage 0 or A), who are not candidates for surgery. The main method for tumor ablation is image-guided, percutaneous radiofrequency ablation which achieves tumor necrosis by the induction of high intra-tumoral temperature. Additional ablation methods include but are not limited to microwave ablation, cyroablation, ethanol injection and external-beam radiotherapy.

Transarterial therapies include but are not limited to transarterial chemoembolization (TACE) and selective internal radiation therapy (SIRT). Transarterial therapies are recommended for patients with intermediate stage HCC (BCLC stage B).

TACE entails intraarterial infusion of a cytotoxic agent, immediately followed by embolization of the vessels that feed the tumor.

SIRT is based on the intraarterial infusion of microspheres with the radioisotope yttrium-90. There is no microembolic step. The radiation emitted by the yttrium-90 is responsible for the anti-tumor activity.

SIRT and TACE have similar objective response rates of about 52.5%.

Systemic therapies include but are not limited to sorafenib, lenvatinib, regorafenib, and cabozantinib. These treatments are recommended for patients who have advanced HCC (BCLC stage C) or stage B and progression with transarterial therapies.

Sorafenib, lenvatinib and regorafenib are inhibitors of multiple kinases. Sorafenib is the standard of care for late stage HCC. Recently, lenvatinib and regorafenib have been shown to be as efficacious and safe as sorafenib. Cabozantinib, an inhibitor of receptor tyrosine kinases, has recently been shown to improve survival with manageable side effects.

Lastly, immune based therapies for HCC are emerging including tremelimumab, an inhibitor of CTLA-4, nivolumab, a PD-1 immune checkpoint inhibitor, and pembrolizumab, also a PD-1 inhibitor.

Patients who are treated at the early stages of HCC (stages 0 and A) have an estimated survival of greater than five years. Patients treated at the intermediate stages of HCC (stage B) have an estimated survival of two years. Those patients treated at the advanced stage of HCC have about 8-13 month estimated survival.

See generally Villanueva 2019.

As shown herein when a patient is diagnosed with HCC at an early stage, he or she has better treatment options available to him or her, as well as a great increase in survival time.

Thus, the current disclosure provides methods for providing treatment to a subject who has been diagnosed with HCC using the disclosed 3-smRC signature with or without the additional use of AFP levels, at an early stage, wherein the treatment includes surgical therapies, including but not limited to resection and liver transplantation, tumor ablation, and immune based therapies. The distinct advantage to this method is that these patients have an increased survival time of five years or more as compared to two years or a year or less with other treatment methods.

The method may further include a step of a confirmatory detection and/or diagnosis of HCC in the subject including further tests or procedures including but not limited to an ultrasound, the detection of alpha-fetoprotein, magnetic resonance imaging (MRI), computed tomography (CT), biopsy or combinations thereof.

### Kits

It is contemplated that all of the methods and assays disclosed herein can be in kit form for use by a health care provider and/or a diagnostic laboratory.

Reagents for practicing methods for the detection of the three, small unannotated non-coding RNAs having the nucleotide sequences SEQ ID NOs: 1, 3 and 5 and thus for the detection and/or diagnosis of HCC can be incorporated into kits. Such kits could include primers and/or probes specific for the three small unannotated non-coding RNAs, reagents for isolating and/or purifying exosomes from a sample, extracting RNA from the exosomes, additional reagents for detecting the three small unannotated non-coding RNAs, reference values or the means for obtaining reference values in a control sample for the three small unannotated non-coding RNAs, and instructions for use, including the formula used to calculate the risk of HCC. The kits could further include reagents for purifying AFP from a sample and reagents for detecting AFP in a sample as well as reference values or the means for obtaining reference values in a control sample for AFP.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of ordinary skill in the art should, in light of the present disclosure, appreciate that many changes can be made in the specific embodiments which are disclosed and still obtain a like or similar result without departing from the spirit and scope of the disclosure.

### Example 1—Materials and Methods

### Patient Enrollment

For the prostate cancer dataset, de-identified data and biospecimens from human subjects consented under ongoing IRB approved protocols at the Icahn School of Medicine at Mount Sinai (GCO #14-0318, 15-1135 and 10-1180) were collected from prostate cancer patients undergoing prostatectomy. Specifically, biospecimen included, prostate cancer and adjacent prostate specimen from biopsy or prostatectomy, urine, and serum, where applicable. Each of these protocols involves the prospective collection of clinical data (e.g., demographics, baseline characteristics, treatments, and outcomes). Samples for the HCC biomarker discovery and biomarker validation cohorts were collected from consented patients enrolled in an IRB approved protocol to derive new HCC biomarkers from blood (HS-15-00540) or provided by the Tisch Cancer Institute Biorepository (HSM #10-00135) at the Icahn School of Medicine at Mount Sinai. For the HCC biomarker discovery cohort, HCC cases and controls were collected from the same setting as for the validation cohort. Importantly, HCC cases and controls were matched for age, gender, presence of cirrhosis, and etiology. For the case-control biomarker study, three patient populations were included: 1) HCC cases were limited to very early or early stage patients according to the BCLC classification (Villanueva 2019) (i.e., stages 0 or A). All HCC patients were treatment-naïve at the time of blood sampling; 2) Patients with liver cirrhosis or different forms of chronic liver disease (CLD) at risk for HCC as per clinical practice guidelines (European Association for the Study of Liver 2018), but without radiological evidence of HCC at the time of blood collection; 3) Patients with benign liver nodules (e.g., hemangioma) without chronic liver disease. HCC diagnosis was made according to the criteria of the European Association for the Study of the Liver (EASL) (European Association for the Study of Liver 2018). In a subset of patients (n=30) sequential blood samples were available after these patients had received HCC treatment. Response was assessed according to modified RECIST criteria (Lencioni and Llovet 2010). Liver cirrhosis was diagnosed based on histology, or non-invasively through combined transient elastography, imaging or laboratory evidence of liver dysfunction and portal hypertension. Patients with concurrent malignancies were excluded. Small RNA sequencing data from patients with other (non-HCC) malignancies were downloaded from exRNA atlas (exrna-atlas.org/, including n=100 colon cancer, n=6 pancreatic adenocarcinoma, and n=36 prostate cancer patients, respectively). Patients with concurrent malignancies were excluded.

Sample Collection and Separation of EVs from Human Plasma

For the prostate cancer dataset, human serum was collected using BD Vacutainer blood collection tubes (i.e., serum separation tubes). First, whole blood was centrifuged at 2,000 g for 30 minutes at 4° C. followed by another centrifugation of the serum at 12,000 g for 45 minutes at 4° C. to remove larger EVs (e.g., microvesicles and apoptotic bodies). The supernatant was carefully transferred to ultracentrifugation tubes (Beckman coulter, thick wall polypropylene tube, Cat #355642) and ultracentrifuged for two rounds in at 110,000 g for 2 hours at 4° C. The pellet was finally resuspended in 1 ml PBS and stored at −80° C. for further analysis. EVs from human urine was collected with the above-mentioned protocol.

For the HCC biomarker discovery and validation dataset, peripheral venous blood was collected in EDTA containing vacutainer (BD Vacutainer), stored on ice, and processed within 4 hours of collection. On the day of collection, two centrifugation steps were performed to separate plasma from other blood components and minimize cellular debris from the final isolate. First, whole blood was centrifuged at 1,600 g for 10 minutes at 4° C. followed by another centrifugation of the plasma at 16,000 g for 10 minutes at 4° C. to remove larger EVs (e.g., microvesicles and apoptotic bodies). The supernatant was then stored at −80° C. until the ultracentrifugation was performed. For this, samples were thawed on ice and 0.5-1 mL of plasma was diluted in approximately 25 mL PBS and centrifuged at 120,000 g for 2 hours at 4° C. with a Type 50.2 Ti Fixed-Angle Titanium rotor (Beckman Coulter, k-factor=69). Isolates were directly used for RNA extraction (see below) or resuspended in PBS and stored at −20° C. until further analysis.

### EV Characterization

After differential ultracentrifugation, the PBS-resuspended isolate was evaluated with transmission electron microscopy (TEM) in a Hitachi 7000 transmission electron microscope operating at 80 kV. Briefly, equal volumes of the isolate and 3% glutaraldehyde were mixed and kept at room temperature for 1 hour. Two μl of osmium tetroxide was added to the mixture and incubated at room temperature for 1 hour. The solution was then transferred to formvar coated TEM grids and observed under the electron microscope. To estimate the size and concentration of the isolate, nanoparticle tracking analysis (NTA) was conducted on a NanoSight NS300 (Malvern Instruments Ltd, Malvern, UK) and analyzed the samples with the NTA 3.2 software (Malvern). For this, PBS-resuspended isolates were diluted 1:50 in PBS.

For immuno-labeling of the isolate, western blotting was performed for the intracellular marker TSG101 and Exoview™ analysis for colocalization of tetraspanins CD9, CD63, and CD81. For western blotting, protein concentration was quantified (Bradford assay, Biorad) and 20 μg of protein were separated by sodium dodecyl sulfate-polyacrylamide electrophoresis under reducing conditions and transferred to PVDF membranes (Life Technologies). Unspecific binding sites were blocked with 5% nonfat dry milk and membranes were incubated with mouse monoclonal TSG101 antibody (ab83, Abcam) at 4° C. overnight followed by goat anti-mouse secondary antibody (A0447, Agilent Technologies) for 1 hour at room temperature. Chemiluminescence was detected using the ECL™ Prime Western Blotting System (RPN2232, GE Healthcare). Exoview™ experiments were carried out on an ExoView™ R100 imaging platform (NanoView Bioscience). With the Exoview™ Tetraspanin kit, 35 μl of PBS-resuspended isolate was incubated overnight on a microarray chip which has been functionalized with antibodies against CD9, CD63, CD81, plus IgG negative control to detect EVs expressing these surface markers. After washing off unbound particles, chips were stained with fluorescence-conjugated antibodies against CD9 (Alexa 647) or CD81 (Alexa 555) to identify subpopulations based on maker profiles. Analysis was done with the NanoViewer 2.4.5 (NanoView Bioscience).

### RNA Extraction, Small Library Preparation and Next-Generation Sequencing

For the prostate cancer dataset, total RNA was extracted from the serum/urine bump fraction (nanoDLD, serum only) or UC isolates or bulk tissue using the Total Exosome and Protein Isolation Kit (Invitrogen 4478545) by following the protocol. For the HCC biomarker discovery and validation dataset, RNA was extracted from the UC isolate on the same day of ultracentrifugation using the miRNeasy Plasma/Serum kit (Qiagen) according to the manufacturer's recommendations including the spike-in C. elegans miR-39 miRNA mimic and stored at −80° C. until further use. EV-RNA quantitation and quality were assessed on a 2100 Bioanalyzer Instrument (Agilent) with the RNA 6000 Pico Kit (Agilent). Indexed Illumina Small RNA libraries were prepared with the SMARTer® smRNA-Seq Kit (Clontech Laboratories, Inc.) and sequenced on an Illumina HiSeq 4000 (prostate cancer dataset) or HiSeq2500 (liver cancer dataset) platform.

### Small RNA-Seq Read Trimming

The SMARTer™ smRNA-Seq kit yields reads that were flanked on the 5′ end by a leading triad of three bases from SMARTer™ template switching activity, and on the 3′ end by the Illumina adapter and extra bases from the oligo dT (which are exactly 15 bp in length). Cutadapt (Martin 2011) was used to remove the first 3 nucleotides of all reads, specify the homopolymer adapter sequence AAAAAAAAAA (SEQ ID NO: 9) to remove along with any sequence 3′ of it, and finally discard all reads that are smaller than 15 base pairs long after these filters were applied. The exact command used, as recommended by the (strand-sensitive) SMARTer™ smRNA-Seq kit, is

cutadapt -m 15 -u 3 -a AAAAAAAAAA (SEQ ID NO: 9) input.fastq>output.fastq

Therefore, the set of initial small RNAs were at least 15 bp long and were trimmed from positions 1-3 and also from the oligo dT 3′ through to the adapter. It is noted in passing that although template switching at low frequencies can add more than 3 nucleotides to the 5′ end, there was no trimming any further on the 5′ end.

### Deconvolution Analysis

EV carrier deconvolution analysis was performed as a post-processing step to the standard exceRpt pipeline (Rozowsky et al. 2019), which was applied to the entire HCC smRC discovery dataset (n=15). The output of exceRpt is collated (using mergePipelineRuns.R from github.com/rkitchen/exceRpt) to form summary data of count matrices for key annotated, noncoding RNA biotypes (piRNA, circRNA, miRNA, tRNA counts), aggregated QC data, adapter sequence data, and diagnostic plots. At this point their deconvolution algorithm was applied on the summarized data. Briefly, this consists of two key stages. In the first stage, constituent cargo profiles are estimated using a modified version of a methylation deconvolution technique in Onuchic et al. 2016. Next, deconvolution is performed using the Read Counts or RPM sample profiles from the exRNA Atlas and the per-sample proportion enrichments of each profile are estimated.

Table 2 displays the relevant information from the top four selected smRCs. Subsequent RT-qPCR validation revealed that smRC_125851 had relatively poor discriminatory power between HCC and CLD, so it was removed. The remaining three smRCs were profiled via RT-qPCR in the early ‘HCC biomarker validation’ cohort and subsequently used to create an early HCC risk function using penalized logistic regression.

### Reverse Transcriptase Quantitative Polymerase Chain Reaction (RT-PCR)

Custom TaqMan® Small RNA Assays were designed to target the three smRC clusters (ThermoFisher) and purchased a catalog TaqMan® miRNA Assay against cel-miR-39-3p (ThermoFisher) to target the spike-in miRNA mimic Three μ.1 of extracted EV-RNA were used for reverse transcription (RT) to cDNA with the conventional TaqMan™ MicroRNA Reverse Transcription Kit (ThermoFisher) and target-specific RT primers, followed by quantitative real-time PCR according to the manufacturer's protocol. Raw ct values of smRCs were corrected against ct values of the spike-in (ΔCt) and normalized to the average ΔCt of all controls (ΔΔCt). Overall, the turnaround time from blood sampling to final test results can be achieved in less than 12 hours.

smRC Overlap with Known RNA Biotypes

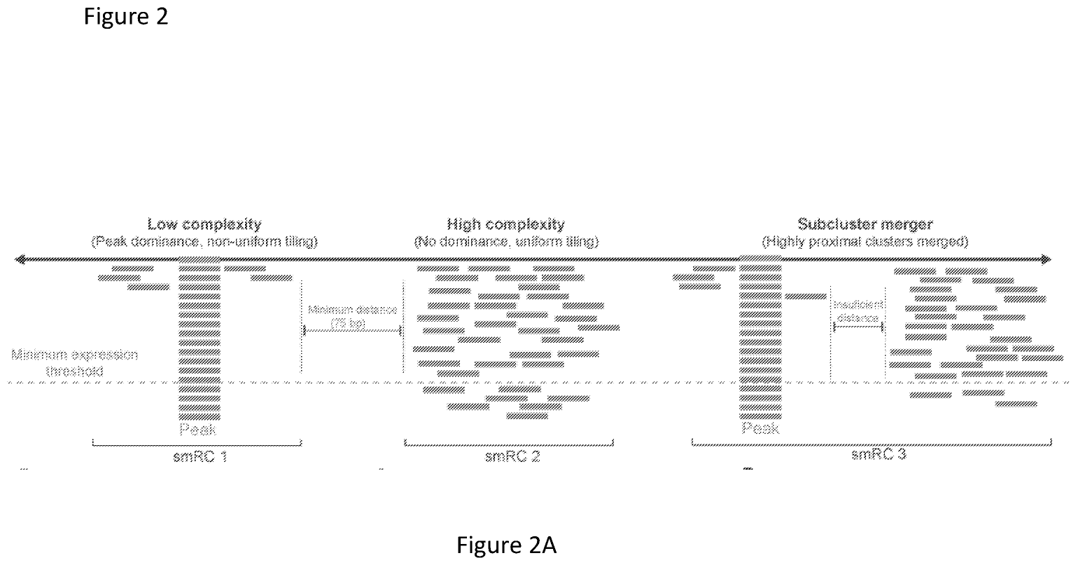

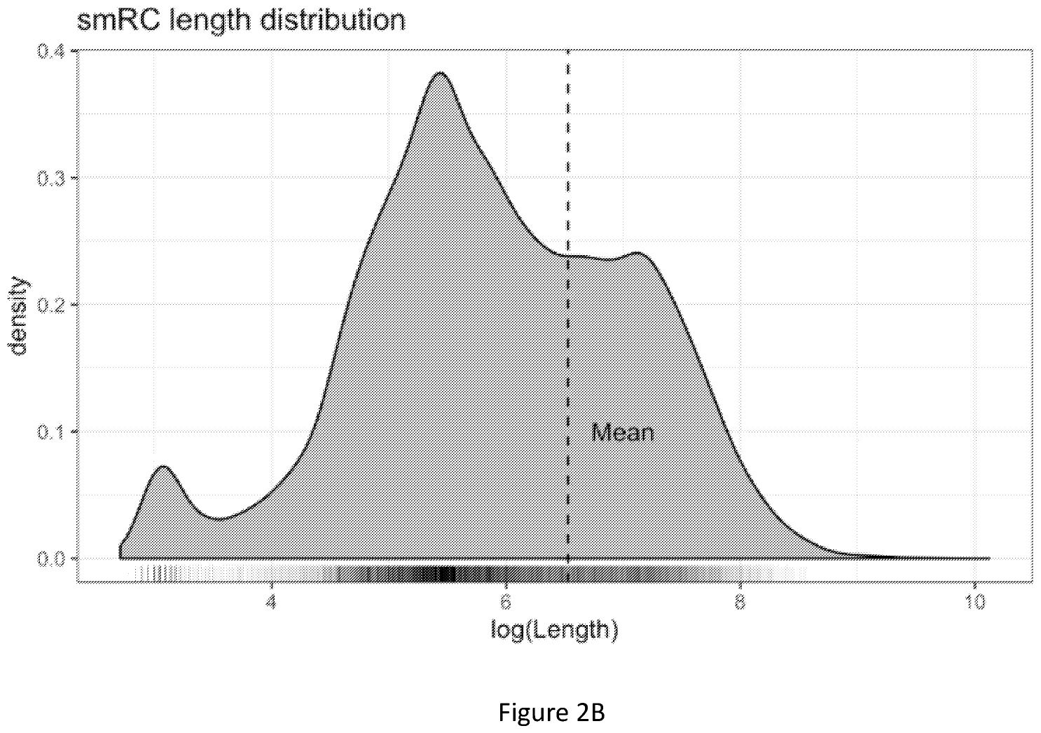

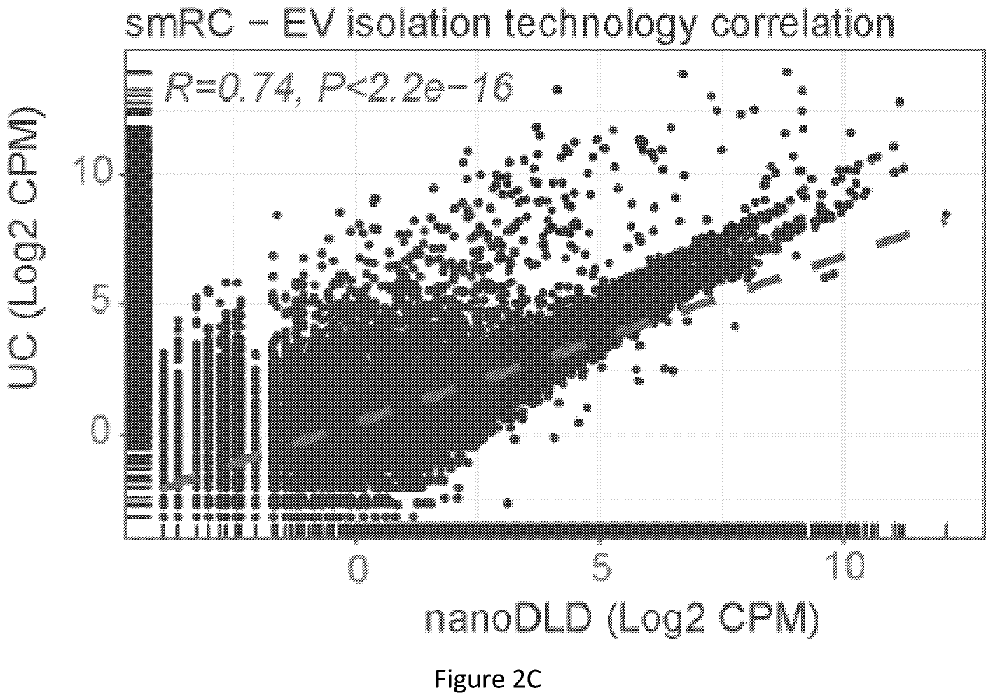

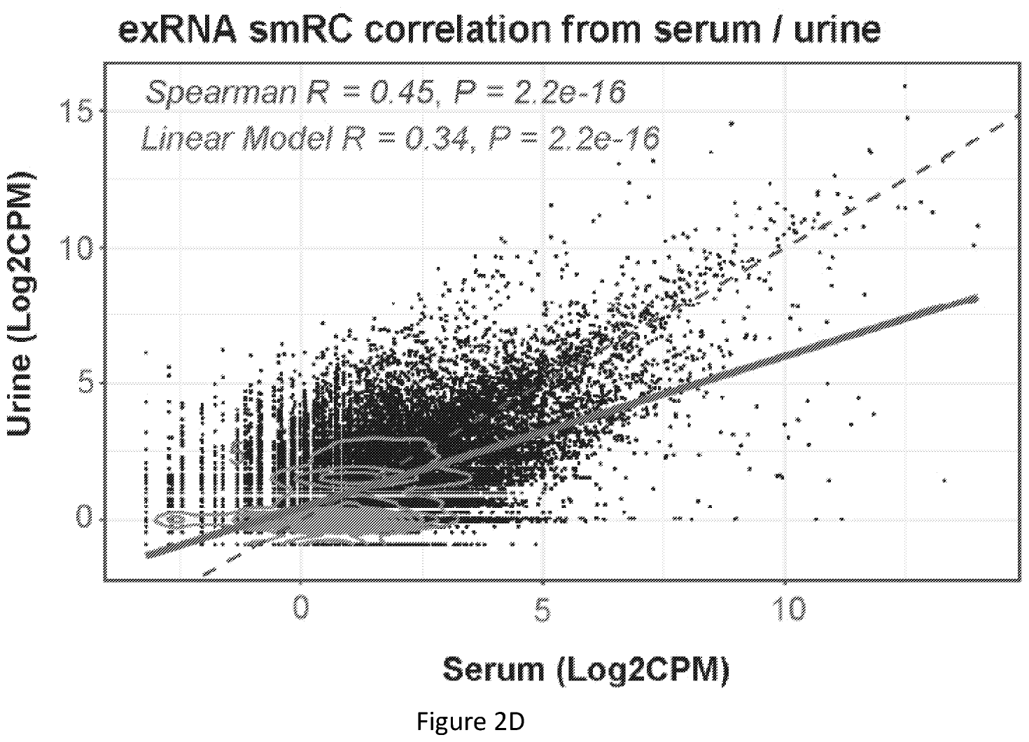

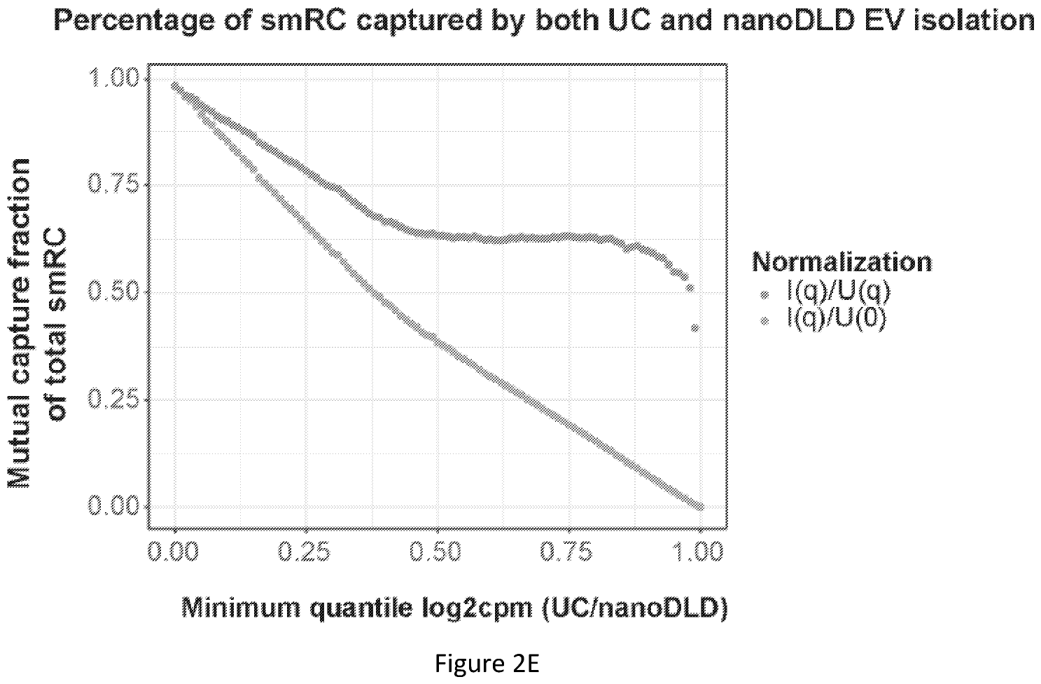

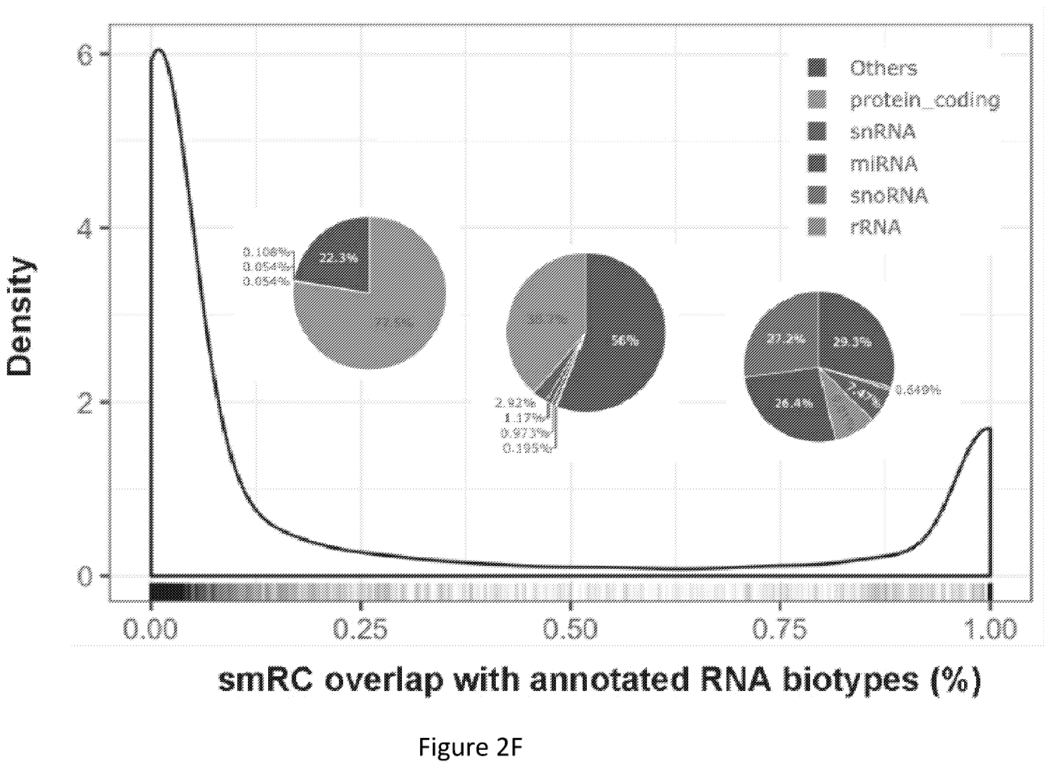

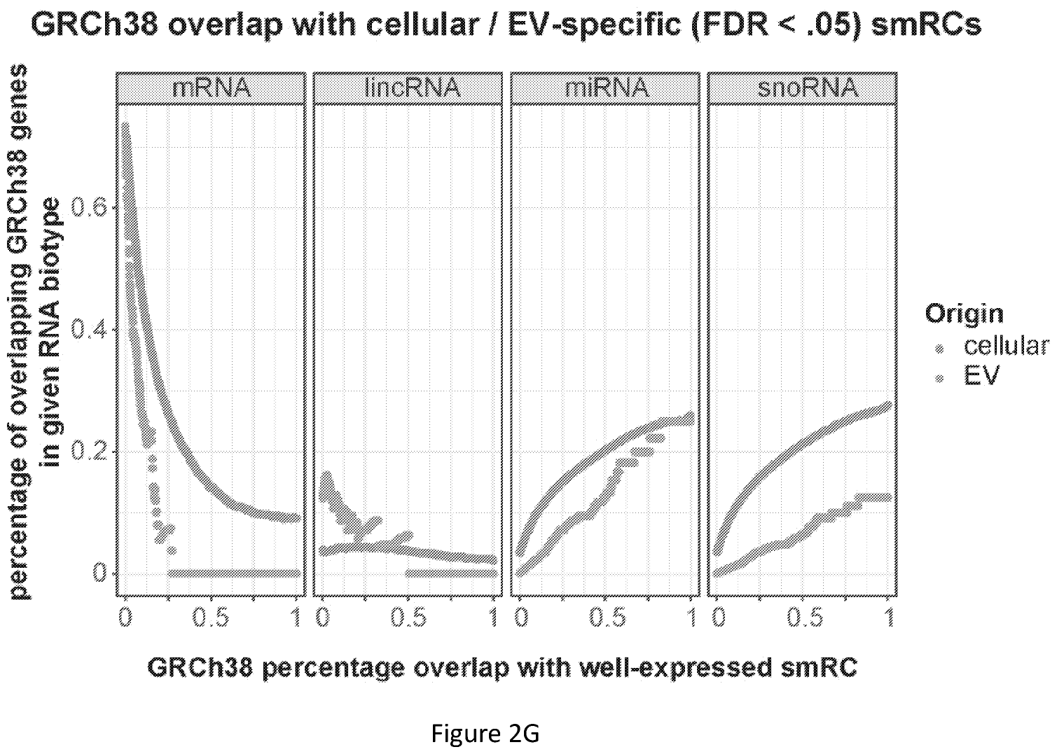

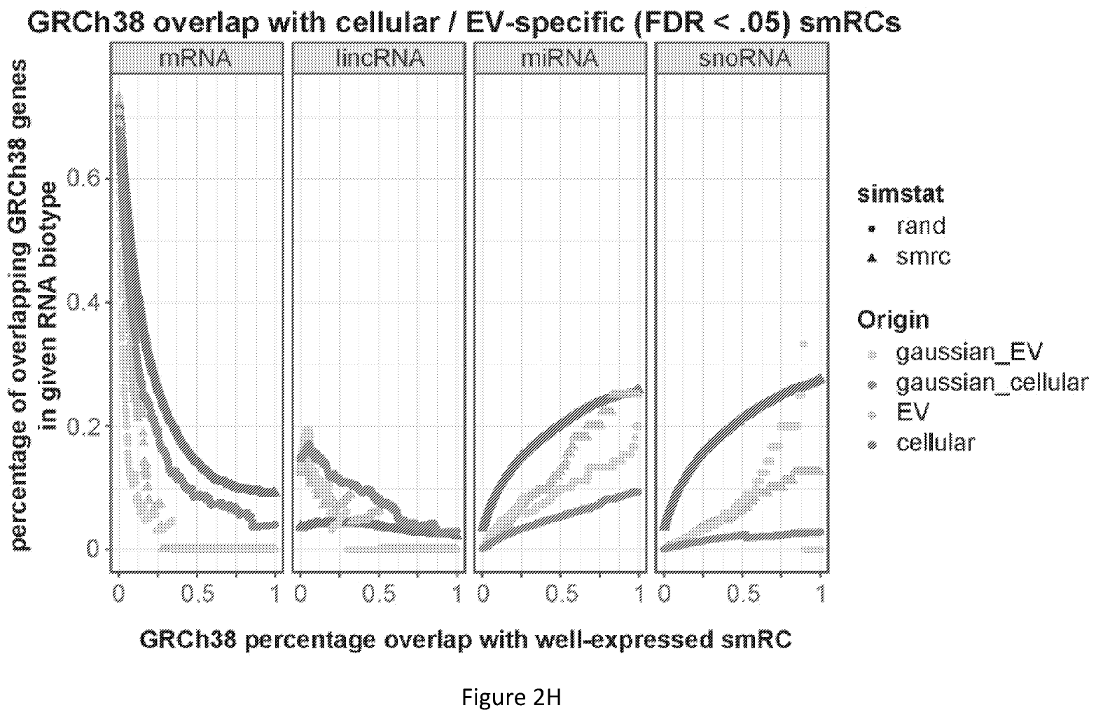

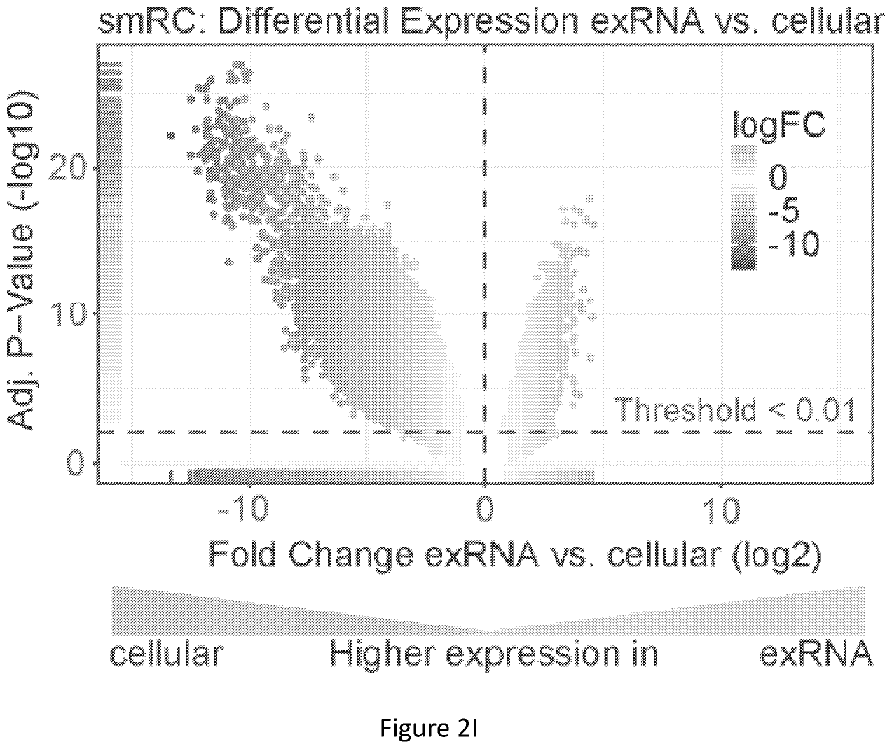

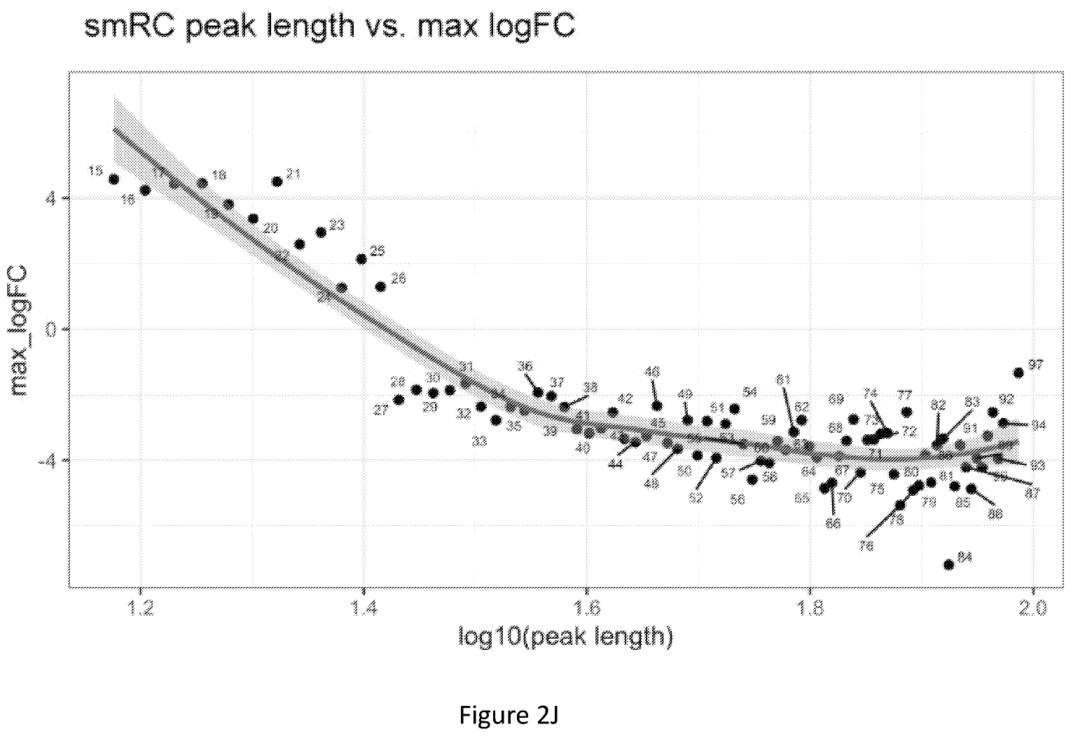

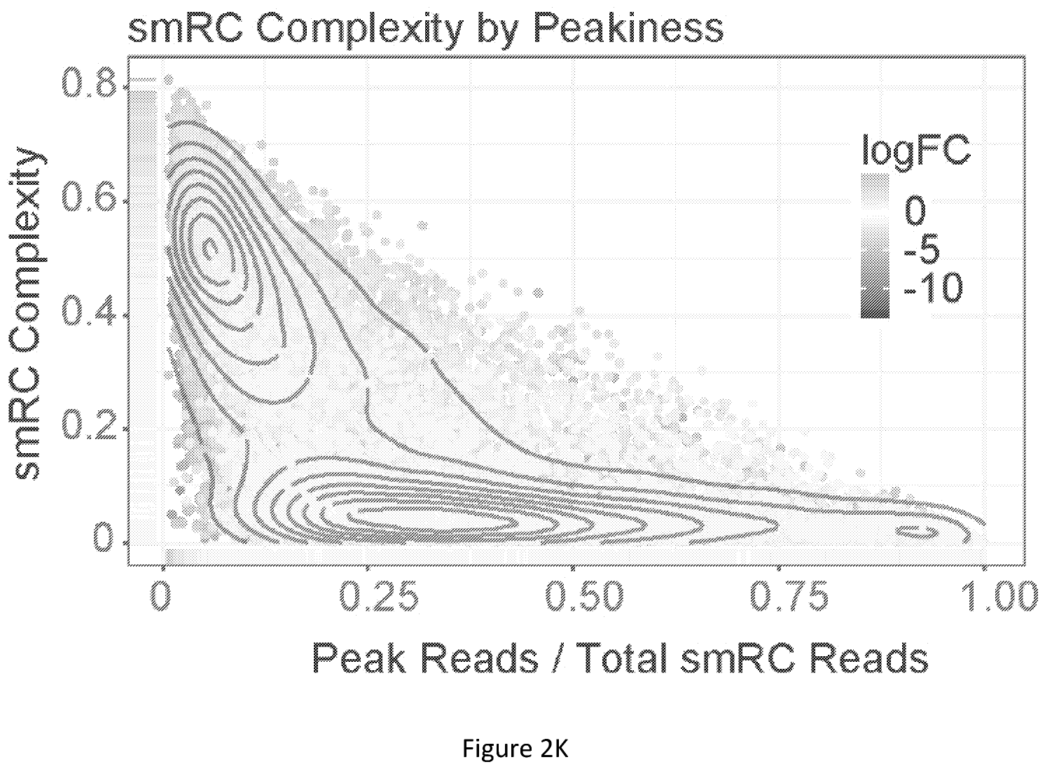

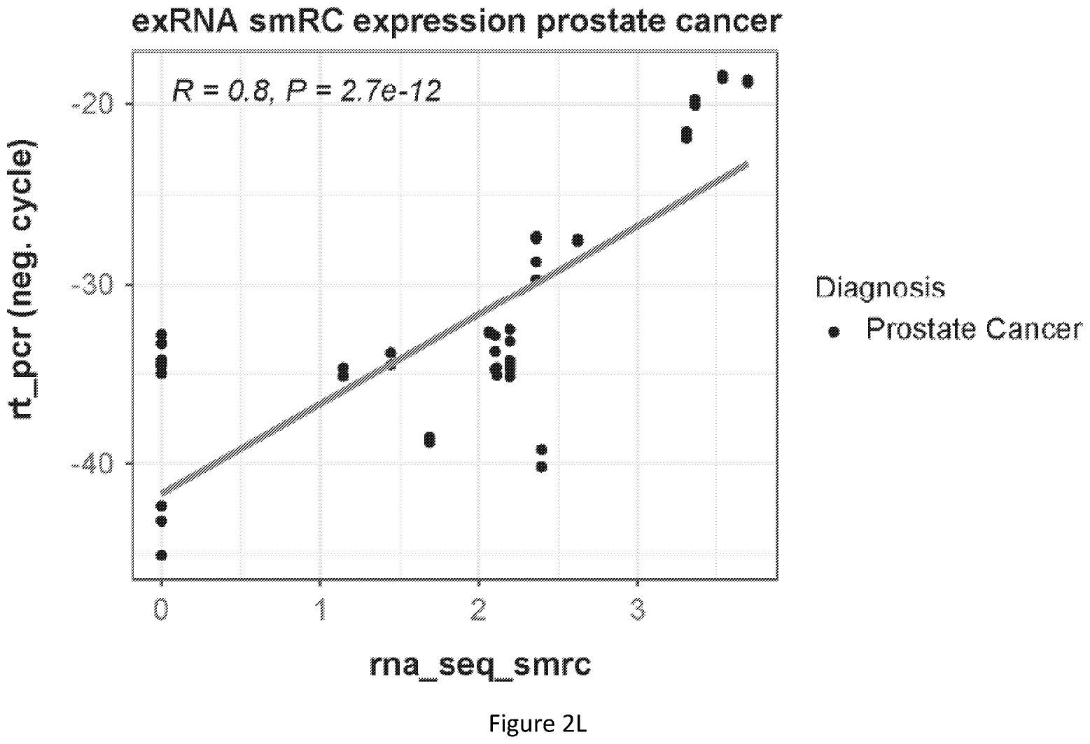

It was next investigated if well-expressed exRNA and cellular smRCs preferentially captured (enclosed) any key known RNA biotypes, as one would expect with both exRNA and cellular smRCs for miRNA for example, and to what extent they do so across all key biotypes. Indeed, for a specific RNA biotype the smRC capture percentage (i.e., whether or not the smRC completely or only partially enclosed the RNA biotype) was first computed. Then, for a particular smRC capture percentage, it was asked how frequent a particular RNA biotype was among all biotypes. FIG. 2F shows the relative breakdown of RNA biotypes at several extremal points of the smRC capture percentage (1%, 70%, 100%), where plainly miRNA, snoRNA, snRNA, and other small RNA were preferentially completely captured (i.e., they are the dominant RNA biotypes with capture overlap˜1) by smRCs compared to mRNA, which are dominantly grazed (i.e., protein coding biotype is dominant for capture overlap <<1). In other words, as expected, when a smRC completely or mostly encloses a known RNA biotype, it is most likely a small RNA and very unlikely a protein-coding RNA. Indeed, plotting the RNA biotype frequency across all exRNA and cellular smRC capture overlap percentages separately for mRNA, lincRNA, miRNA, and snoRNA, yields (FIG. 2G). It was found that exRNA smRCs dominantly partially captured (grazed) mRNA at most to about 25% of the mRNA transcript, and never captured more, while cellular smRCs tended to overlap more protein-coding mRNA and can actually completely enclose mRNA. Similarly, using FIG. 2G, one can conclude miRNA were preferentially completely enclosed by both exRNA EV and cellular smRCs at the same rate, at most 50% of a lncRNA was captured by an exRNA smRC, and snoRNAs were preferentially completely enclosed by cellular smRCs compared to exRNA smRCs. Taken together, when smRCs do enclose known RNA biotypes they can either do so predominantly partially (as with mRNA and lncRNA) or predominantly completely (as with miRNA, snoRNA, and other small RNA), with key differences in the statistics observed between exRNA and cellular smRCs.

Finally, one can ask if these overlap properties are principally driven by the number and relative size distributions of exRNA and cellular smRCs (as opposed to a genuine property of small RNA accumulation in exRNA and cells). Randomly generating genomic regions with the same number of regions, and exRNA and cellular smRC size distributions (masking for repeat regions and centromeres), the above overlap computations were repeated using a Kolmogorov-Smirnov test to assess if the underlying distributions of overlaps and capture percentages are the same within sampling noise. It turned out that all pairwise (x=smRC, y=random) Kolmogorov-Smirnov tests with two sided alternative hypotheses were highly significant, especially for lncRNAs, indicating that the exRNA and RNA biotype specific overlap patterns are not solely attributable to the size distributions (or number) of smRCs. As Table 3 illustrates for the two separate one-sided KS tests, interesting trends emerged: for mRNA, both exRNA and cellular smRCs tend to overlap more exons than expected by random simulation; for lncRNA, exRNA smRCs overlap than expected more while cellular smRCs overlap much less; for miRNA, both exRNA and cellular smRCs overlap far more than expected by chance; for snoRNA, cellular smRCs overlap far more than expected while exRNA smRCs have slightly more evidence for relative depletion.

In summary, exRNA smRCs overlap known RNA biotypes in a non-random fashion, and when they completely or almost completely enclose a biotype it is overwhelmingly likely to be a known small RNA biotype, as opposed to similar but distinct trends for cellular smRCs. To aid in interpretation and comparison, FIG. 2H also includes the simulated fractional overlap curves. However, as FIG. 2F demonstrates a significant fraction of exRNA smRCs are well-expressed from unannotated genomic regions.

### Data Analysis

Following the guidelines of the Early Detection Research Network by the National Cancer Institute (Pepe et al. 2001), a case-control biomarker study for early detection of HCC was conducted. Based on the largest meta-analysis on surveillance for HCC, sensitivity of the current gold-standard for HCC surveillance (i.e. abdominal ultrasound and AFP) is 63% for early stage tumors (Tzartzeva et al. 2018). This study was powered to detect an increase in sensitivity from 63% to 75% and specificity from 83% to 95%. Given an alpha of 0.05 and a power (1-B) of 80%, the number of samples needed to detect this difference based on asymptotic normal distribution theory (Pepe 2003) was 101 cases (early HCC) and 71 controls (patients at high risk of HCC). For descriptive statistics, continuous variables were reported as median and categorical variables as counts and percentages. The Fisher's exact test and the Student's t-test were used to compare differences between categorical and continuous variables, respectively. Pearson's or Spearman's correlation coefficient were computed for correlation of continuous variables as indicated. Boxplot center line shows median, box limits show upper and lower quartiles, whiskers show 1.5× interquartile range, and points represent outliers. Error bars represent the 95% confidence intervals

The analysis of the case control biomarker study to test the performance of the 3-smRC signature for early detection of HCC was limited to early stage HCC (n=105) and controls at risk for HCC (n=85) to represent the optimal population of interest (Marrero et al. 2018; Pepe et al. 2001).

Further, a full logistic regression model was built to predict early HCC/CLD diagnosis. Penalized maximum likelihood techniques, bootstrap and cross-validation were used to estimate and control for model optimism, RT-qPCR batch plate effects and over-fitting. Also, the positive and negative predictive power estimates of the 3-smRC early detection signature were rigorously computed.

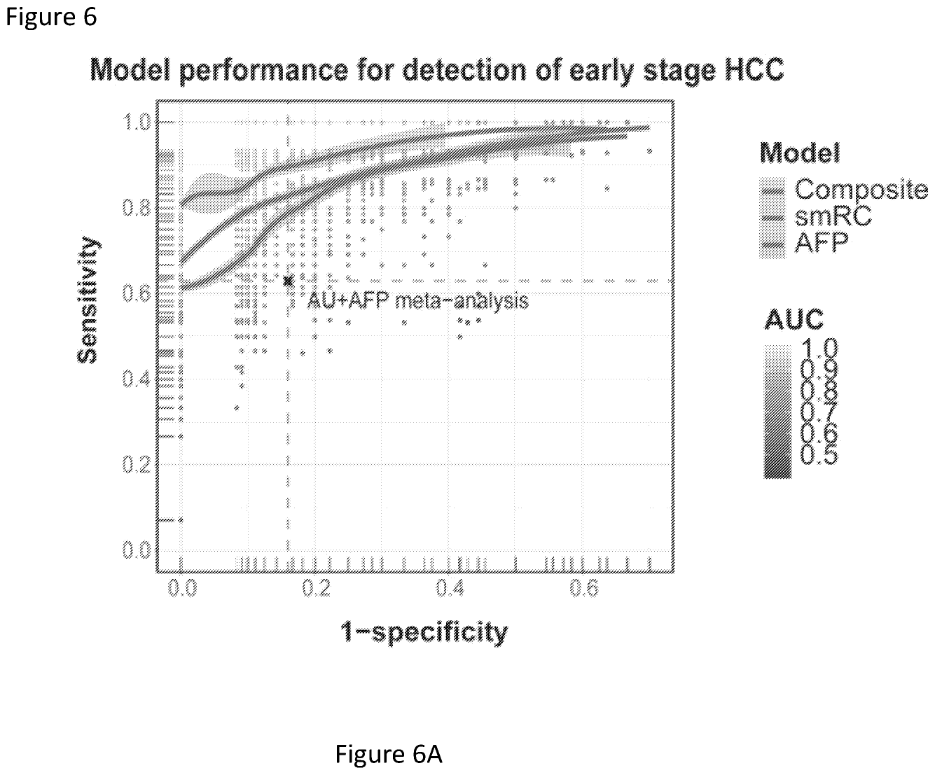

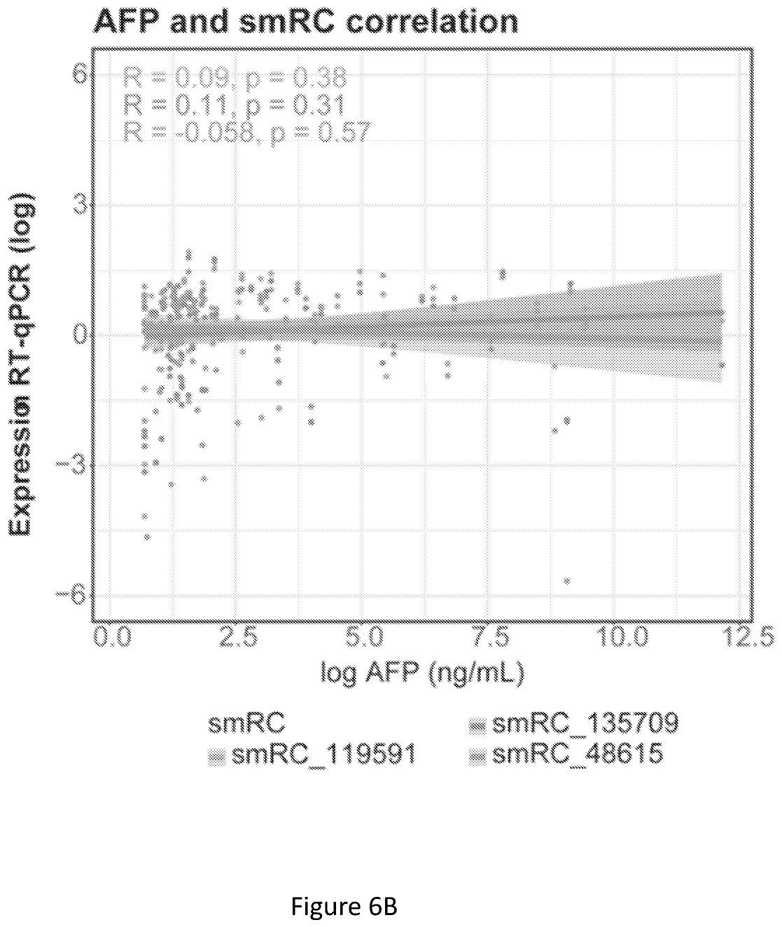

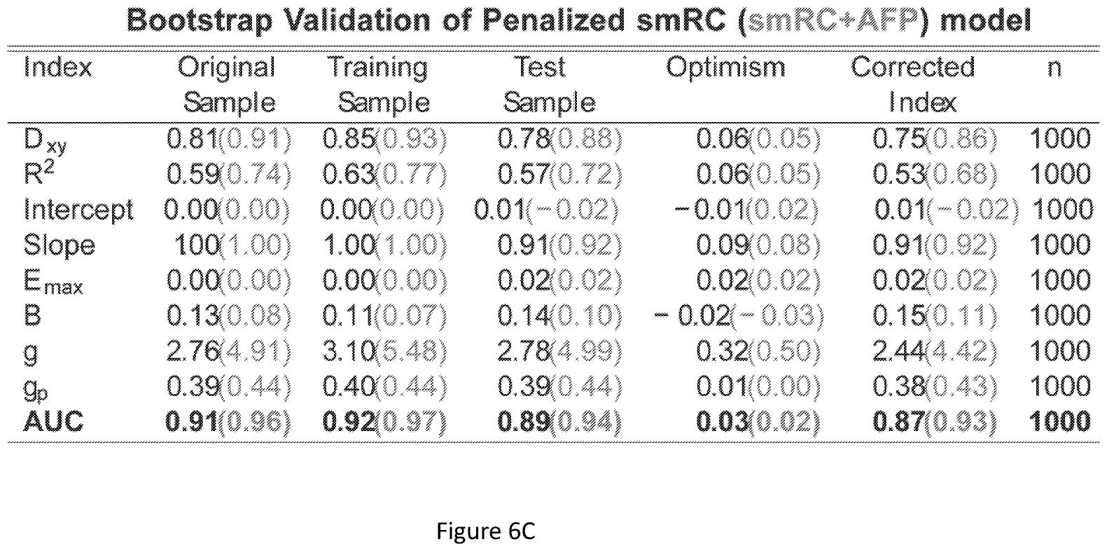

We computed a number of indices of model performance, discrimination measures, and calibration measures under bootstrap resampling (n=1000), in order to demonstrate model performance and estimate generalization error by averaging performance across bootstrap resampling. In the first row, the key measure of discrimination Somers' Dxy is the rank correlation between the observed and predicted response values, which in the case of logistic regression for a binary response reduces to simply Dxy=2(c−½), where c is Harrel's c-statistic and equal to the AUC of the ROC for the early-HCC, CLD prediction. In the case of the smRC model, we immediately deduced that the bootstrap adjusted AUC is ½+⅜=⅞=0.875. Modest adjusted modified R2˜0.52 was observed, combined with bootstrap-adjusted slope and intercept indicating modest and acceptably low over-fitting. Relatively bootstrap-adjusted low Emax (the maximum error in predicted probabilities), modest Brier score (B), very low unreliability index (U), high discrimination (D), high quality (Q=D−U), also indicated a reasonably robust model. Also, the bootstrap adjusted total Gini's mean difference for based on the smRC model was a healthy 2.44, which robustly represents typical log-odds differences between early HCC and CLD patients predicted by the model. Converting this early HCC log-odds estimate to an early HCC probability prediction, we saw that the typical predicted probability gap between early HCC and CLD patients is 38%. Finally, we computed the partial mean gini-scores of the smRC model predictors and found that the smRCs themselves have by far the largest termwise log-odds compared to any technical variance covariates (e.g., batch). We note in passing that repeated cross-validation gave similar results for Dxy and adjusted Slope (FIG. 6C).

We next repeated the penalized maximum likelihood estimation procedure using a model with both smRCs and AFP readings included, given that a log likelihood ratio test for a AFP term was highly significant (p<1e−8). Computing the same indices of model performance across bootstrap resampling (n=1000), we found dramatically better performance as shown in FIG. 6C, with bootstrap adjusted AUC˜0.93, lower overall error and evidence for overfitting, a much smaller adjusted Brier score of 0.11, and a dramatic increase in the Gini indices such that a typical HCC-CLD predicted probability difference was 43%.

Finally, even though balanced accuracy is not a proper scoring rule, we estimated the maximized balanced accuracy landscape by subjecting the smRC logistic regression model for HCC risk to a cross-validation repeated 1000 times (i.e., a random 85% training, 15% testing split repeated 1,000 times) and computing maximizing sensitivity and specificity on the test ROCs. We found strong evidence to suggest that sensitivity ˜85% and specificity 91% for smRC-only models, while for smRC+AFP models we found sensitivity ˜86% and specificity ˜99%. All statistical analyses were conducted on Rstudio (R version 3.5.0).

### Example 2—Isolation of EVs from the Patient Samples

This study was based on three independent cancer EV datasets: 1) a prostate cancer cohort, termed the ‘smRC characterization’ cohort, to define and study the properties of smRCs in exRNA (n=9 patients, total of 41 samples); 2) a HCC ‘biomarker discovery’ cohort (n=157 patients) to identify differentially expressed smRCs between HCC patients and controls with chronic liver disease, and patients with non-HCC malignancies to test the HCC-specificity of the biomarkers; and 3) an independent HCC ‘biomarker validation’ cohort (n=209 patients, total of 281 samples, including 42 patients with replicates and 30 patients with longitudinal samples before and after HCC treatment) to confirm their clinical utility in a phase 2 biomarker study for detection of early stage HCC. In total, the study included 479 samples from 375 patients.

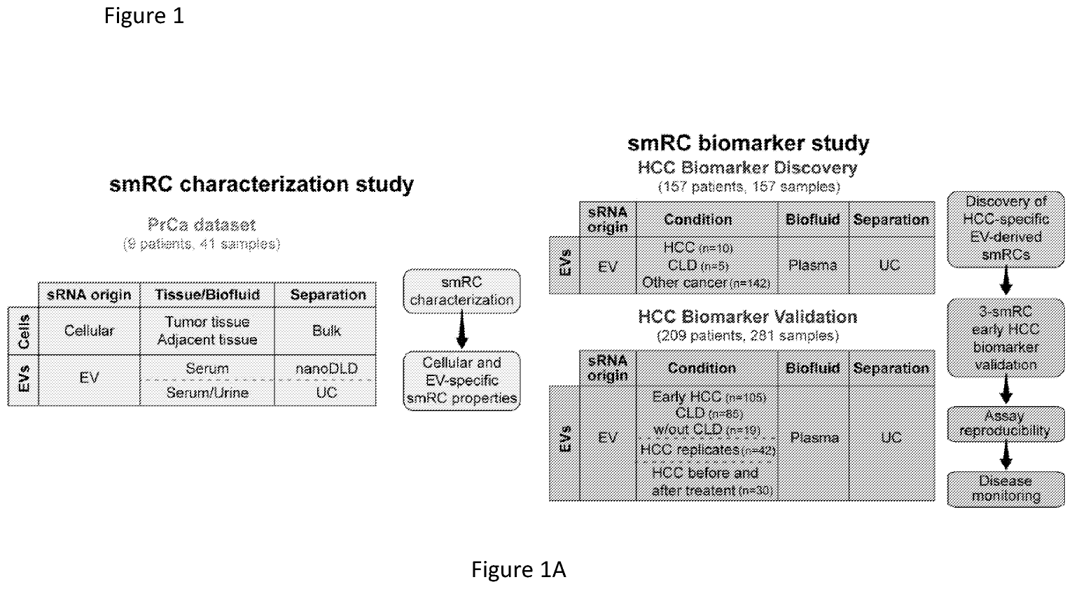

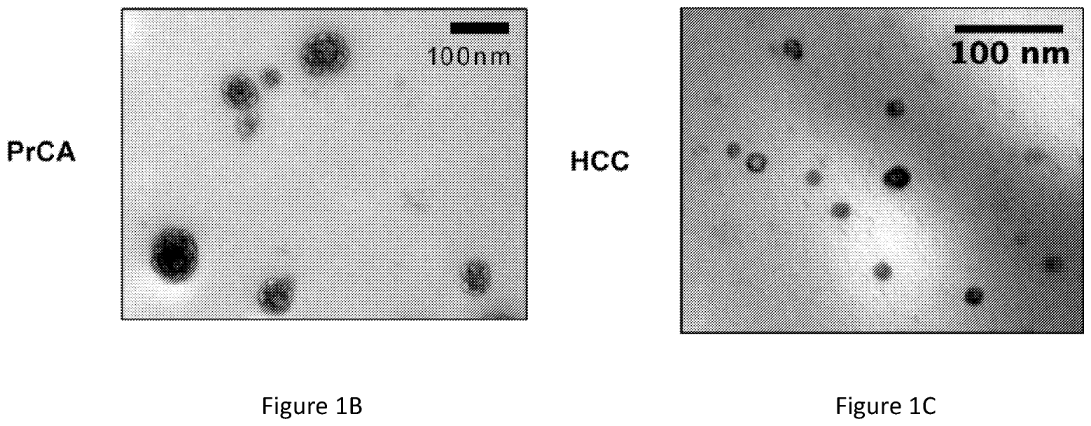

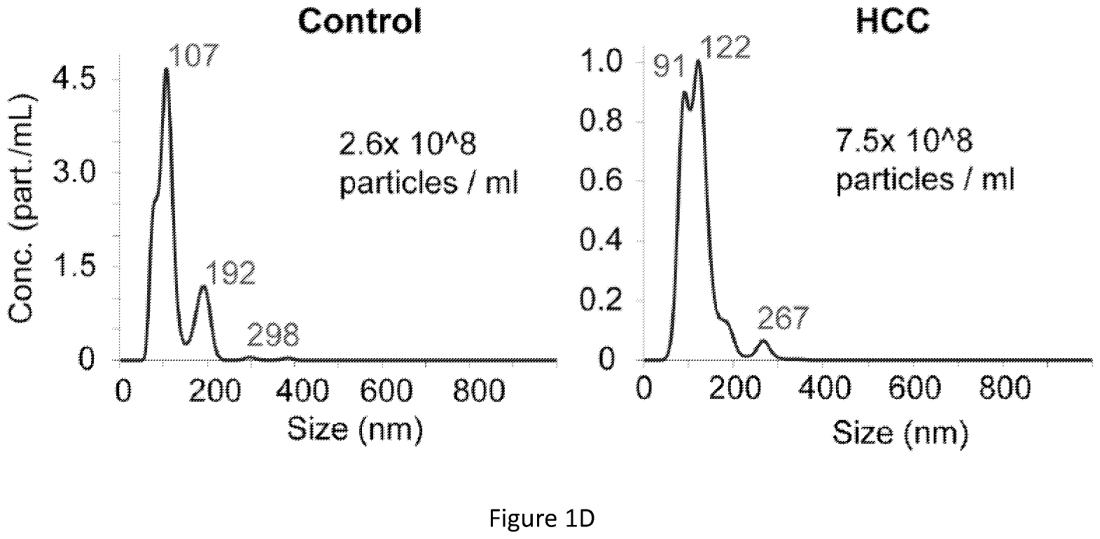

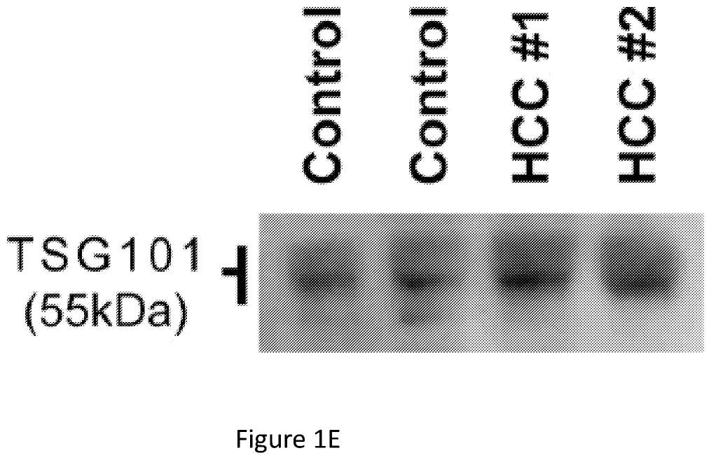

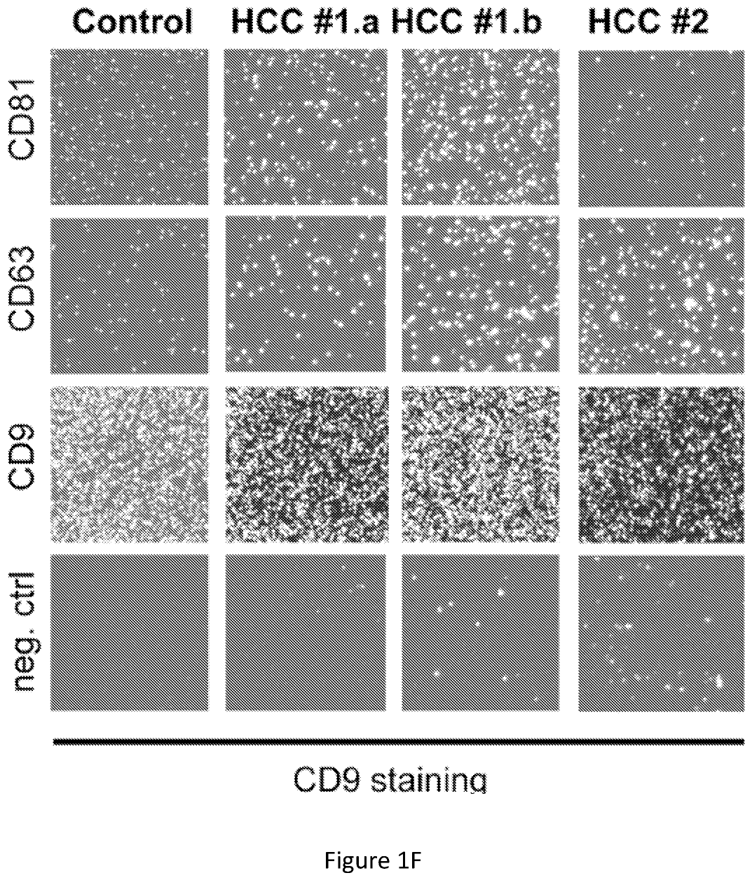

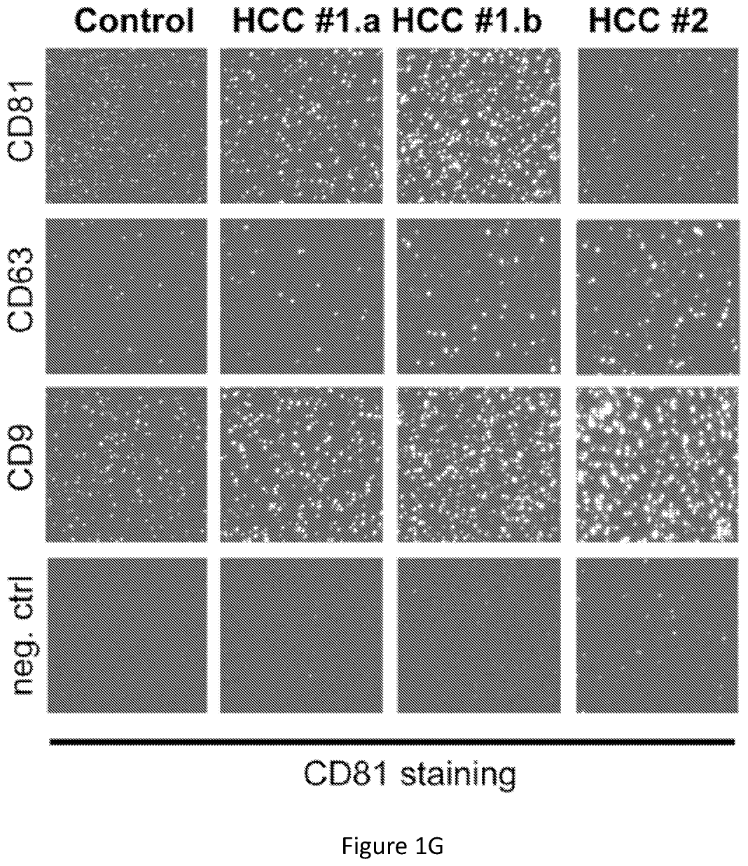

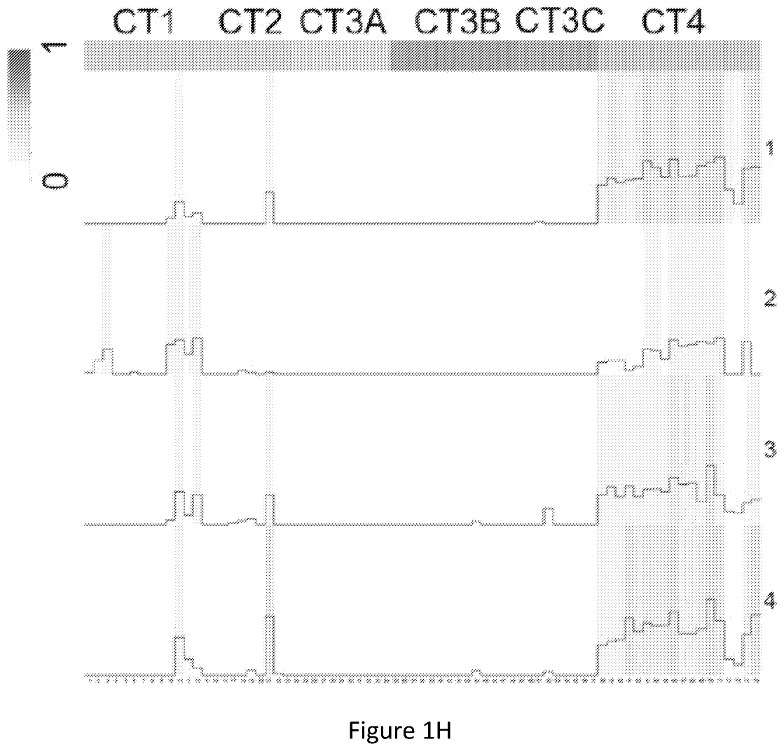

In all cohorts, differential ultracentrifugation (UC) was employed to isolate EVs, following the recommendations of the International Society of Extracellular Vesicles (Thery et al. 2018) for quality assessment of EV isolates. Specifically, transmission electron microscopy, nanoparticle tracking analysis, immuno-labeling with Western Blotting for intracellular (i.e., tumor susceptibility gene 101 protein, TSG101) and Exoview™ for transmembrane (i.e., tetraspanins CD9, CD63, CD81) vesicle proteins was used. This confirmed enrichment for small EVs (median size of 120 nm) with compatible morphology and expression of typical markers (Mathieu et al. 2019; Kowal et al. 2016). See FIG. 1. This suggested an enrichment for small EVs (median size of 120 nm on NTA) with compatible morphology on NTA and TEM (FIG. 1B-D), and expression of typical markers for small EV populations with a dominance of CD9/CD81 and CD9/CD63 co-expression, and a paucity of CD63/CD81 co-expression (FIGS. 1E-G).

Additionally, for the smRC characterization cohort in prostate cancer, EVs were isolated from a subset of patients (n=5) using the ‘lab-on-chip’ technology nanoDLD (Wunsch et al. 2016) (DLD) in both urine and serum. Purely cellular sRNA was also isolated from prostate cancer and adjacent non-cancerous tissue of the same patients to quantify EV-isolation technology, biofluid, and EV-specific variance in sRNA profiles respectively.

Part of the prostate cancer dataset has been included in an exRNA-atlas based deconvolution analysis published earlier (Murillo et al. 2019). An independent analysis found that the UC and nanoDLD isolation methods used herein specifically isolate low (cargo type 1) and variable (cargo type 4) density vesicles with minimum contamination from lipoproteins (cargo type 2) and argonaute proteins (cargo type 3B). For this study, the same computational deconvolution analysis for the HCC ‘biomarker discovery’ dataset was performed to determine carrier types and found that cargo type 4 was preferentially enriched (FIG. 1H). In fact, cargo type 4 is associated with vesicles in the 60-150 nm size range, which were purified consistently with nanoDLD, and also the lowest-density OptiPrep fractions 1-3 from serum and plasma (Murillo et al. 2019). Cargo type enrichments associated with low density vesicles, lipoproteins, AGO2-positive ribonucleoproteins (RNPs), and AGO-2 negative RNPs were significantly depleted (FIG. 1H).

Together, these results confirmed the successful enrichment of small vesicles from a variety of biospecimens of prostate cancer and HCC patients.

### Example 3—Identification and Characterization of smRC

Small RNA sequencing was performed on the prostate cancer smRC characterization and the HCC biomarker discovery cohorts in order to define clusters of contiguous genomic regions with sufficient alignment coverage (termed ‘small RNA clusters’, smRCs). This allowed the capture of the known heterogeneous genome-wide expression of clusters of sRNA precursors (Zhang et al. 2010), each of which can give rise to multiple functional sRNA products, by defining clusters (see FIG. 2A).

Adjacent smRCs were merged if they overlapped within some minimal padding threshold (75 bp), and the key properties of smRCs that were defined were: a) entropy (i.e., read tiling efficiency or complexity); b) peak coverage; and c) consensus sequence of each smRC. The set of all smRCs, computed once for all samples, was essentially the paired set of all accumulation loci of sRNA expression and their peak-coverage consensus sequences (which range from 15 to 100 nt in length) and constituted a smoothed de-novo assembled small RNA expression landscape with a standard count matrix.

Key genomic properties of smRCs in the ‘smRC characterization’ prostate cancer dataset were delineated due to the availability of different biological sample types (blood, urine, tumoral and non-tumoral adjacent tissue) and different isolation methods (ultracentrifugation and nanoDLD (Kim et al. 2017; Wunsch et al. 2016). The mean genomic length of smRCs was 674 bp (FIG. 2B), while the mean length of the consensus peak sequence was 20 bp.

In order to profile the maximal coverage and overall distribution of expression within smRCs associated with exRNA, two quantities were defined. First, a ‘peak’ coverage which is simply the ratio of reads in the smRC peak to total smRC coverage, and second, a tiling complexity measure which is the ratio of unique read nucleotide sequences to total smRC coverage. Since almost all small RNAs arise from posttranscriptional processing of larger RNA precursors, the quantification of alignment patterns was largely an empirical task for which measures of maxima (peak coverage) and heterogeneity (tiling complexity) become crucial tools to classify these patterns. smRCs with high complexity are those with uniform tiling and few peaks (see FIG. 2A). It was found that the major contributor of smRC variable expression was RNA origin (with low complexity typical in exRNA-versus high complexity typical of cellular smRC origin, FIG. 2K). Technical reproducibility of smRC quantification included comparing two different EV enrichment methods in serum (UC and nanoDLD), and different biofluid compartments (urine and serum) of the same patients. A high correlation was found between enrichment methods (spearman R˜0.74, p<2.2e−16, FIG. 2C) with over 80% of smRCs detected by both methods above the 20th percentile of expression (FIG. 2E). A modest correlation was found between different biofluid compartments (i.e., urine and serum) using UC (spearman R˜0.45, p<1e−16, see FIG. 2D for self-reproducibility).

These findings provided a novel and unsupervised data-driven view of the entire small RNA landscape, including unannotated genomic regions, and identify smRCs as a novel small RNA biotype with robust detection across biospecimens and enrichment methods.

### Example 4—EV-Derived smRCs are Enriched for Non-Coding Transcripts from Unannotated Regions

Well-expressed smRCs possessed a heteroscedastic count variance profile which facilitated usual differential expression analysis via linear modeling. The total number and magnitude of overexpressed smRCs in cells was significantly higher than in exRNA (FIG. 2I). However, a significant difference was observed in the complexity of smRCs found in exRNA compared to cells and it was found that the major contributor of smRC variable expression was RNA origin (with low complexity typical in exRNA-versus high complexity typical of cellular smRC origin (FIG. 2K). Indeed, the bimodal pattern revealed a clear separation between cellular smRCs, which overwhelmingly have relatively high tiling complexity, and exRNA smRCs that have much stronger evidence for high relative peak coverages. The mean size of the peak within smRCs was slightly higher than the minimal trimmed read length and was significantly different between exRNA-derived and cell-derived (16.5 bp versus 22.6 bp, p<1e−16). exRNA-associated smRCs preferentially overlap unannotated small RNA species compared to cellular smRCs (FIGS. 2F-2H, Table 3).

Finally, the expression of three unannotated smRCs were orthogonally validated using RT-qPCR (FIG. 2L).

These data demonstrated that EV-derived smRCs predominantly present with a small number of highly covered peaks (i.e., low complexity and high peak coverage) compared to cellular-derived smRCs and they preferentially capture non-coding small RNA compared to protein-coding RNA but are also significantly enriched in unannotated genomic regions.

### Example 5— Identification of an HCC-Specific 3-smRC Signature in Plasma EVs

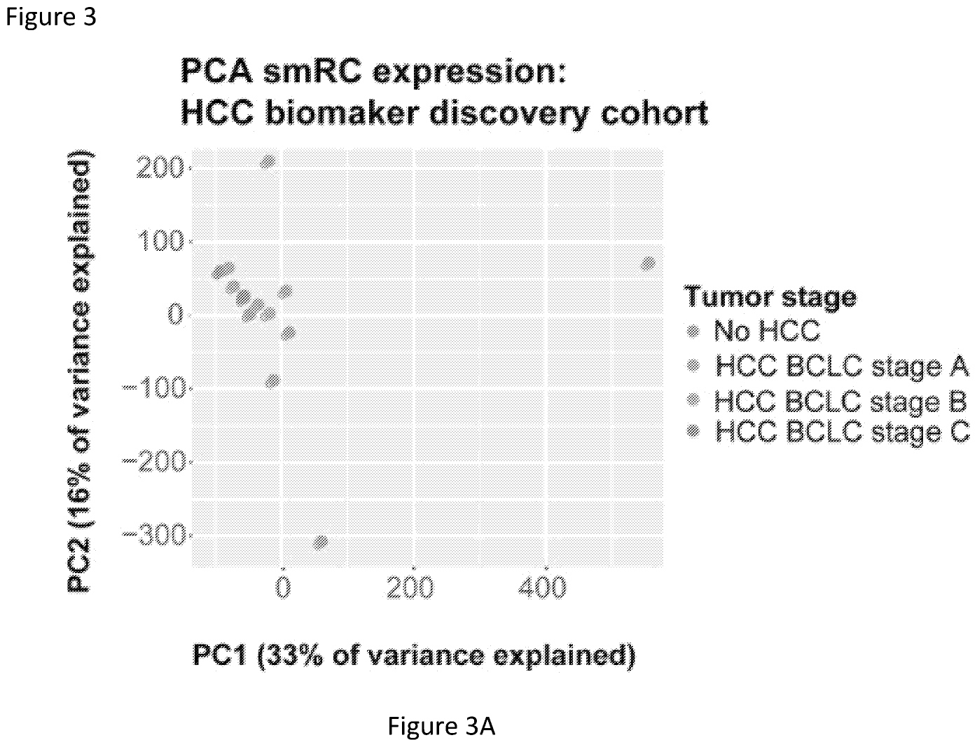

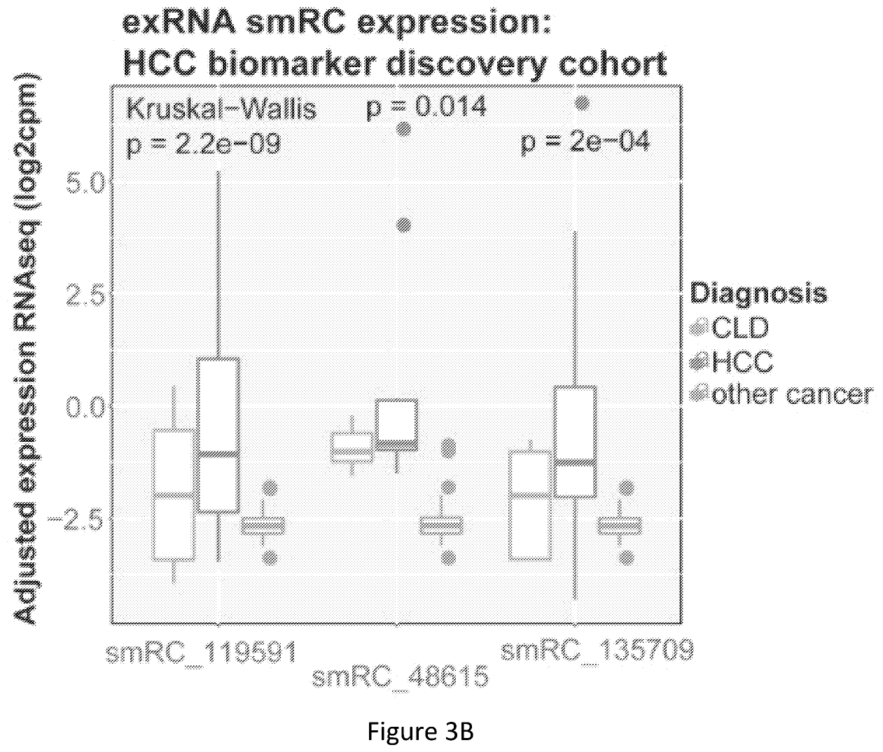

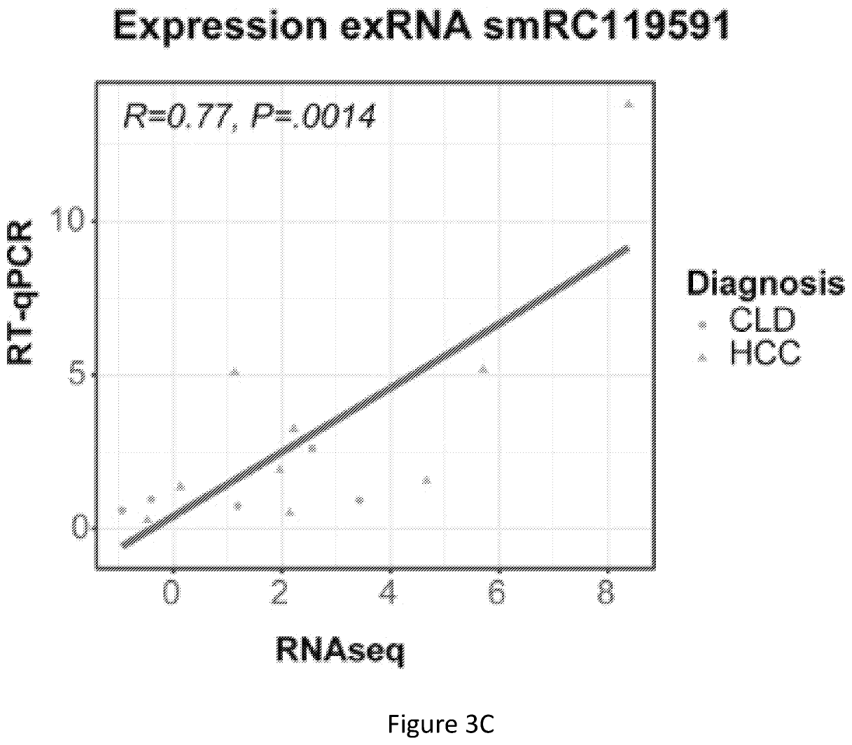

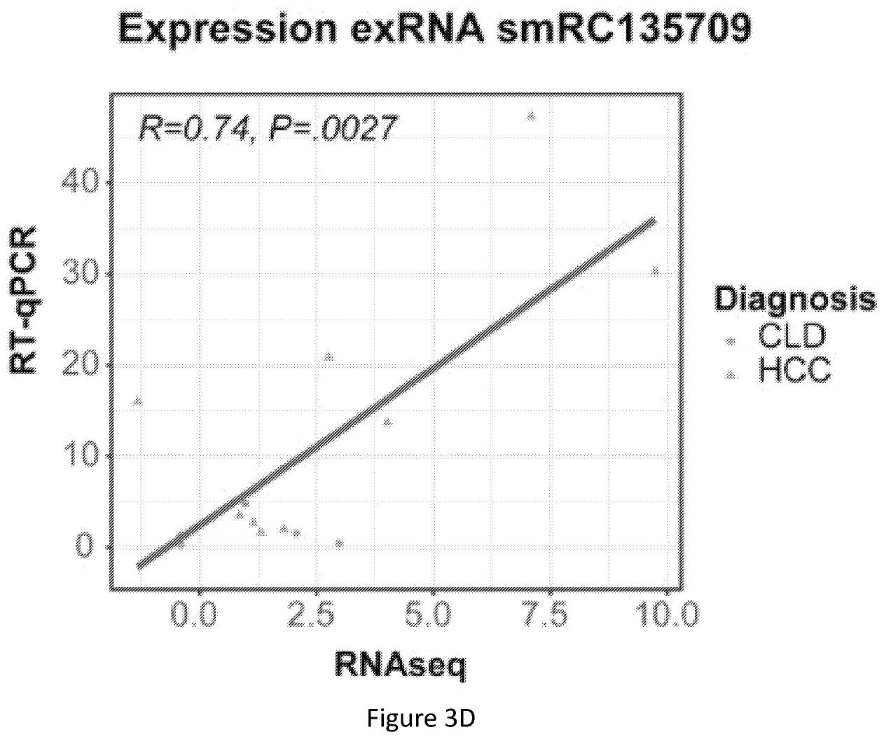

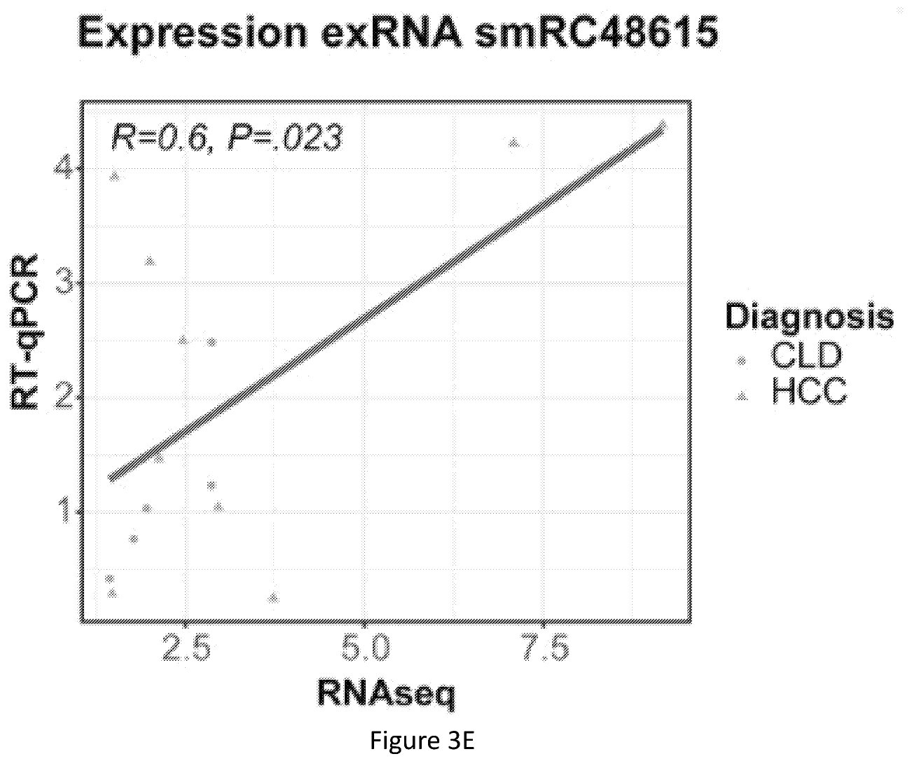

Given their high and technologically independent reproducibility, tractable statistical properties, and unique ability to discriminate concentrations of EV-specific sRNA, the smRC profile was computed of the HCC biomarker discovery cohort of 15 patients, including 10 patients with HCC and 5 controls at risk for HCC matched for age, sex, and etiology of the underlying liver disease (Table 4). It was found that EV smRCs were differentially expressed between HCC and controls. In fact, 250 smRCs were enough to perfectly distinguish them (FIG. 3A). This led to the hypothesis that smRCs could be useful tools for early HCC detection. Thus, three top differentially expressed and low-complexity smRCs were selected for further biomarker analysis and confirmed differential expression between HCC and controls at risk (FIG. 3B). Their differential expression was orthogonally validated in this HCC biomarker discovery cohort using RT-qPCR. Pearson's correlation coefficient was higher than 0.6 for all three smRCs when comparing data from RNA sequencing and RT-qPCR (n=15, p<0.05, FIGS. 3C-3E). The 3 smRCs were located in unannotated regions of chromosomes 3q (intergenic region), 8q, and 10q (intronic region of SGPL1). See Table 2. Additional analysis in a cohort of 142 patients with other malignancies (100 colon cancer, 6 pancreatic adenocarcinoma, and 36 prostate cancer patients) further confirmed their HCC specificity (FIG. 3B).

Altogether, this underscores the great potential of EV-derived small non-coding RNAs for biomarker discovery, particularly the 3-smRC signature for the detection of HCC.

### Example 6—Small RNA Clusters Overexpressed in EVs from HCC Patients Compared to at-Risk Controls

To determine the clinical utility of smRCs in EV, a case control 2 biomarker study was designed following the recommendations from the Early Detection Research Network (EDRN) from the National Cancer Institute (Pepe et al. 2001). In detail, the role of the 3-smRC signature as a novel early detection biomarker in HCC was assessed. Recommended surveillance tools for early HCC detection (i.e., ultrasound and AFP) have low sensitivity (63%) and moderate specificity (83%) (Tzartzeva et al. 2018). Improvement in this area is urgently needed by developing better read-outs of tumor burden and facilitating implementation of surveillance through minimally-invasive, operator-independent tools. Unlike many studies in this setting (Oussalah et al. 2018), only patients with HCC at an early stage (i.e., Barcelona Clinic Liver Cancer classification (BCLC) stage 0 or A) were enrolled, who can be cured with either surgery or ablation (Villanueva 2019). Crucially, the control cohort was the target population for HCC surveillance as defined in clinical practice guidelines (European Association for the Study of the Liver 2018). 209 patients (n=105 treatment-naive, early stage HCC, n=85 control patients with chronic liver disease (CLD) enrolled in HCC surveillance, and n=19 individuals without chronic liver disease) were included. See Table 5. RNA was isolated from plasma-enriched EV and expression analysis of the 3-smRC signature was conducted using qRT-PCR.

The main matching criteria was prevalence of cirrhosis as it is believed this was potentially the strongest variable that could affect the performance of the biomarkers. Differences found in continuous variables related to liver function were not clinically meaningful (Table 5). In fact, when categorizing these variables by clinically relevant cutoffs (i.e., bilirubin at 2 mg/dL, albumin at 3.5 g/dL), no significant differences were observed between the groups. Despite differences observed in age, gender, and etiology, across the groups, expression of any of the smRC was not significantly different for these variables (results not shown).

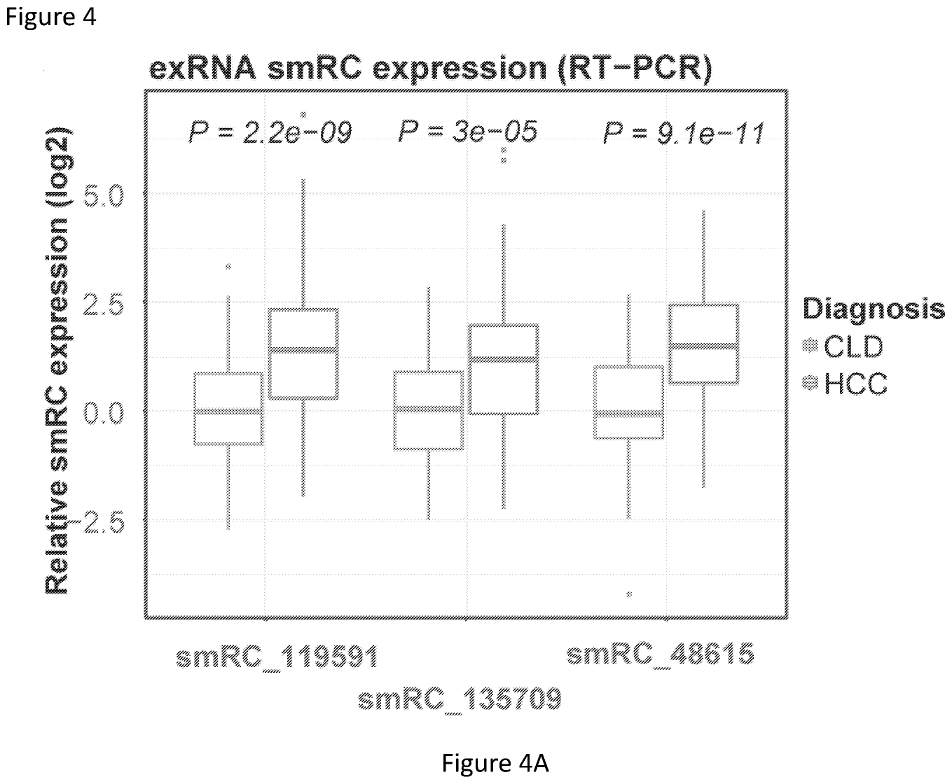

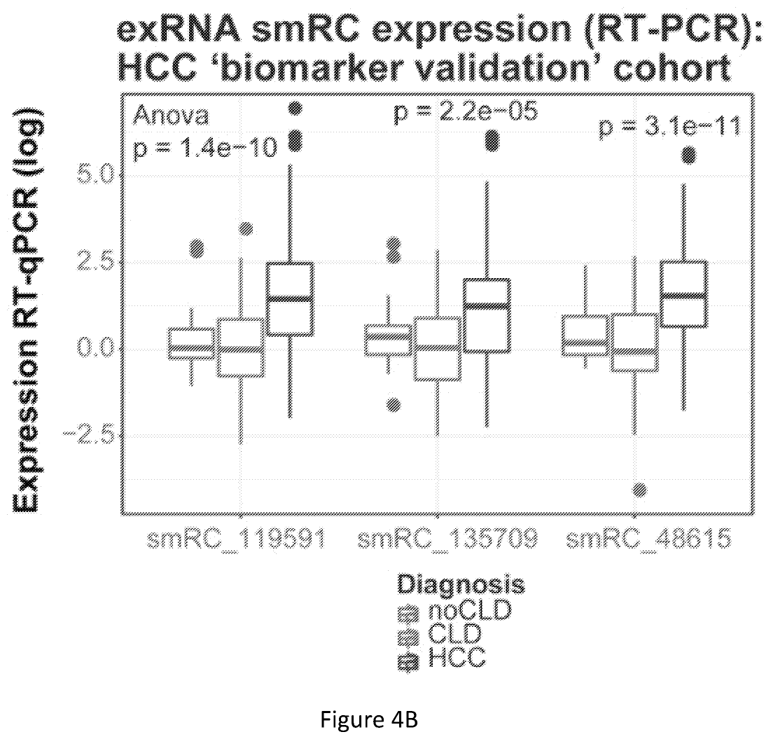

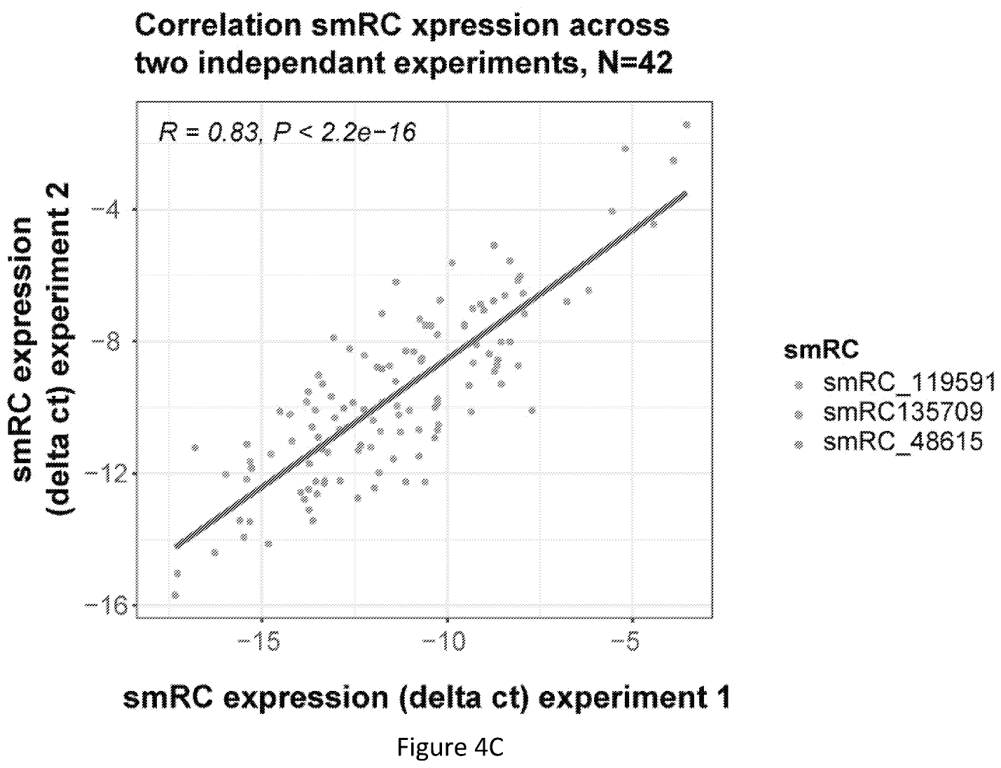

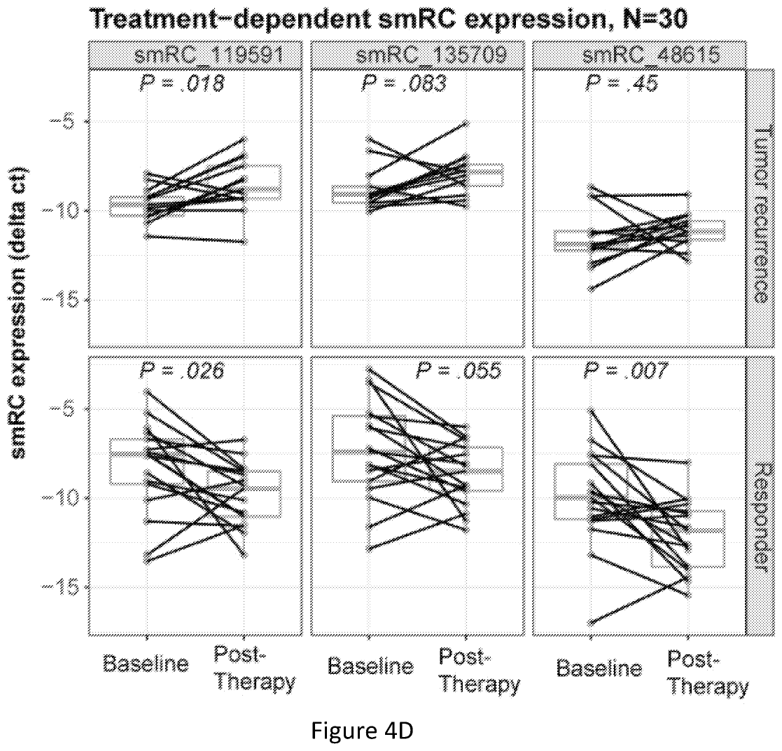

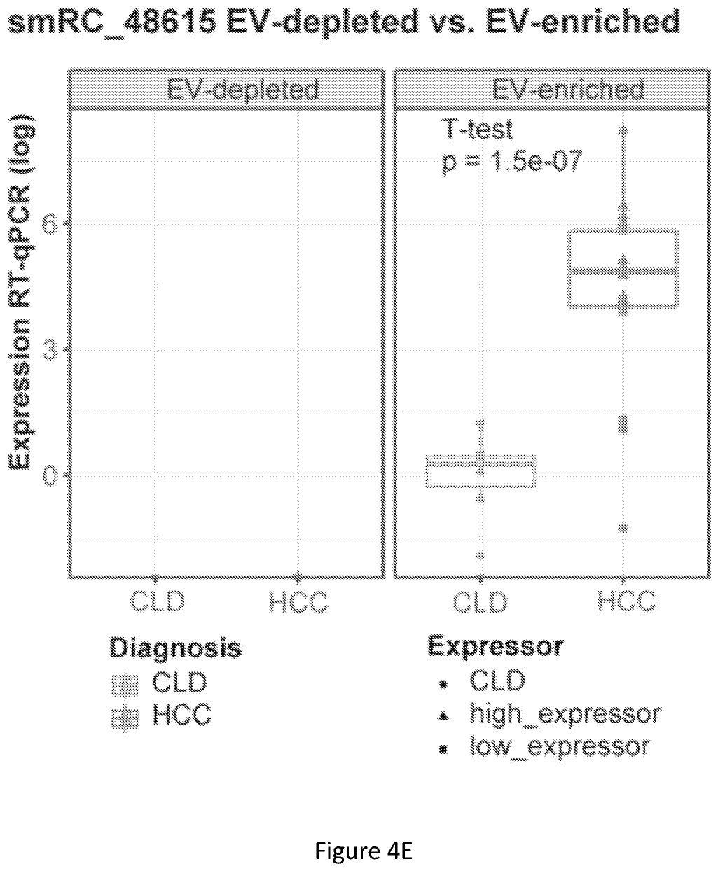

Significant overexpression of the three smRCs in HCC patients was confirmed as compared to CLD controls with RT-qPCR (p<3e−5), (FIG. 4A). See also FIG. 4B for comparison with non-CLD patients.

To confirm the reproducibility of the biomarker analysis, the quantification of the 3-smRC early detection signature was repeated in 42 patients. This included EV isolation from plasma, RNA extraction and RT-qPCR. These two independent experiments yielded a correlation coefficient of 0.83 (p<0.001, FIG. 4C).

Longitudinal analysis in a subset of 30 patients with available sequential blood samples before and after HCC treatment revealed that smRC expression dynamics correlated with tumor response in these patients. In patients without early tumor recurrence after resection (n=13), smRC expression levels significantly decreased compared to baseline (paired t-test, FIG. 4D). The EV origin of the smRC signal was confirmed, as the expression of smRC-48615 was significantly higher using EV-enriched isolates as opposed to EV-depleted plasma (n=30 patients, FIG. 4E).

In summary, the three smRCs with differential expression between early stage HCC and controls at high-risk, who represent the target population for surveillance programs, including replicates and longitudinal samples before and after HCC treatment are shown herein.

### Example 7—a 3-smRC Signature from Plasma EVs Predicts Early Stage Hepatocellular Carcinoma

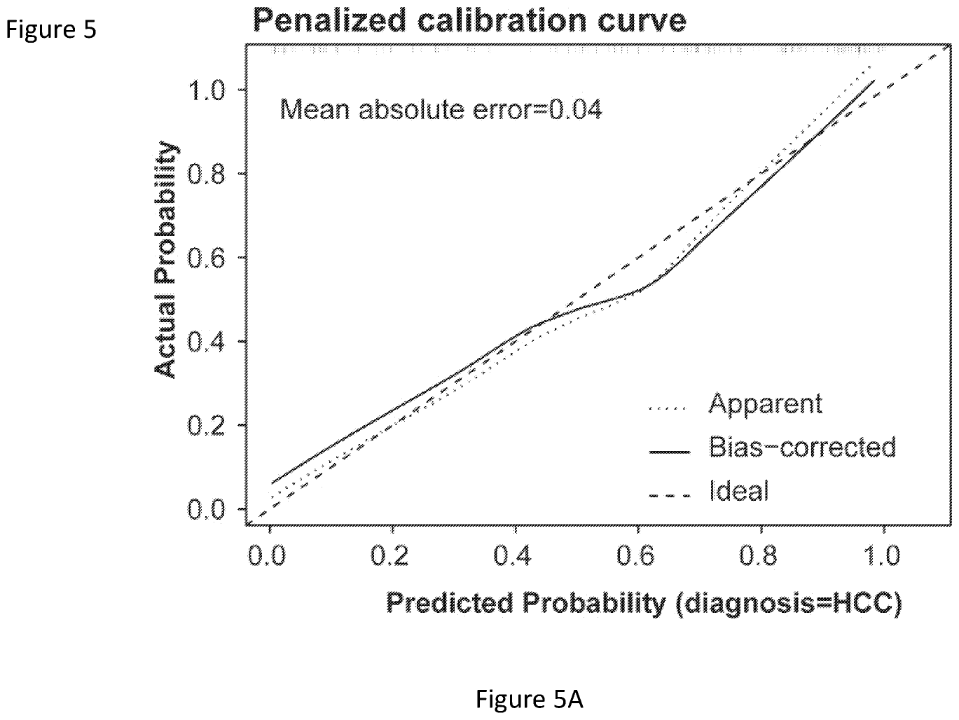

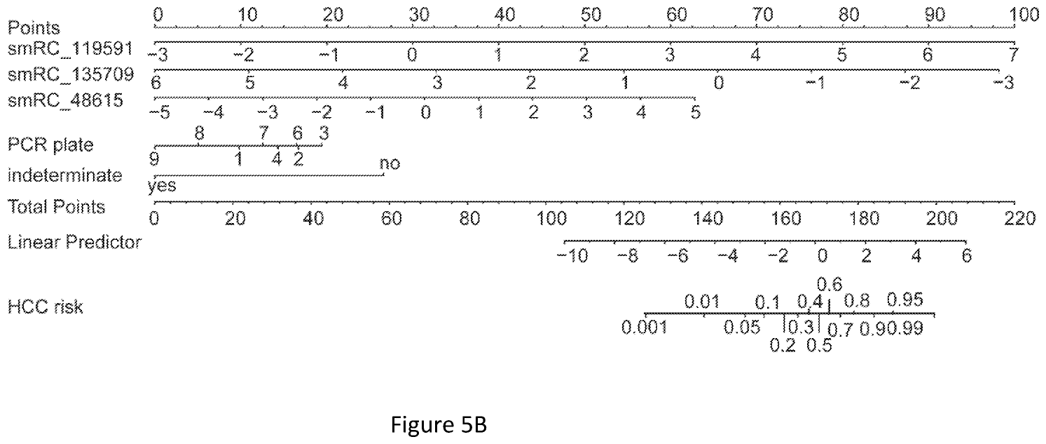

To leverage the collective power of all three smRCs to predict early HCC risk, a parsimonious logistic regression model was built to discriminate between HCC patients and CLD controls (excluding patients without chronic liver disease, non-CLD) using only smRC expression and adjusting for the RT-qPCR sequencing batch effect. This allowed the rigorous testing of whether there was a well calibrated and predictive association between smRC expression and early HCC detection using an appropriate number of effective degrees of freedom in our model. Penalized maximum likelihood techniques, bootstrap and cross-validation were used to estimate and control for model optimism, RT-qPCR batch plate effects, and over-fitting of the 3-smRC early detection signature. The logistic regression model was well calibrated with a low mean absolute error (0.04) to predict early HCC (FIG. 5A), low Brier score (B=0.15), high AUC (0.87), and high Gini mean difference in predicted log-odds between HCC and CLD patients (2.44) adjusted under bootstrap (n=1000) resampling (FIG. 6C). Predicted HCC risk via smRC expression can be visualized via a patient nomogram to provide an individual estimate of HCC risk (see FIG. 5B).

In order to estimate sensitivity and specificity measures at plausible decision points, the logistic regression model was applied to a 85/15 split of the biomarker validation set for training and testing respectively. Averaging over one thousand iterations, an 86% sensitivity and 91% specificity was recovered with a positive predictive value (i.e., true positive rate) of 89% and a false positive rate of 10% on average by maximizing the balanced accuracy of the test ROC curves (FIGS. 6A and 6C). The area under the ROC curve (AUC) for the 3-smRC model was 0.87.

Finally, a likelihood ratio test between an AFP-only early HCC detection model and one incorporating both AFP and the 3-smRC early detection signature showed that the smRCs add significant predictive power to AFP alone (p<0.0001). As expected, AFP levels and expression of the 3-smRC signatures were not correlated (FIG. 6B), which suggested that both capture complementary signals for early HCC detection. Indeed, a blood-based composite model of the 3-smRC signature and AFP yielded an increased AUC of 0.93, lower Brier score of 0.11, and better test performance (85% sensitivity, 100% specificity, positive predictive value of 95%) (FIGS. 6A and 6B)

These data confirmed that the plasma EV-derived 3-smRC signature robustly yields high accuracy in predicting early stage HCC among patients at high-risk, independent of AFP. A composite model including our 3-smRC signature and AFP further enhances its performance.

### Example 8— Extended Phase 2 Biomarker Study

Following the guidelines of the Early Detection Research Network by the National Cancer Institute (Pepe et al. 2001), a population-based case-control phase 2 biomarker study for early detection of HCC is conducted. Based on the largest meta-analysis on surveillance for HCC, sensitivity of the current gold-standard for HCC surveillance (i.e. abdominal ultrasound and AFP) is 63% for early stage tumors (Tzartzeva et al. 2018). We will power this study to detect an increase in sensitivity from 70% to 80% and specificity from 83% to 90%. Given an alpha of 0.05 and a power (1-B) of 90%, the number of samples needed to detect this difference based on asymptotic normal distribution theory (Pepe 2003) is 241 cases (early HCC) and 227 controls (patients at high risk of HCC). Assuming a drop-out rate of 5-10% 250 cases and 250 controls are used to complete the study. Samples come from NIH, Mount Sinai Hospital and external collaborations.

It is expected that results from this larger study will be similar to the results in Example 7.

### Example 9—Comparison of Exosome Isolation/Purification Methods

The performance of three commercially available methods to purify and/or isolate EVs from 40 samples (2 with HCC and 20 controls) is evaluated. The methods are as follows: exoRNeasy Serum/plasma Midi Kit (Qiagen); METMKit (New England Peptide); and ExoQuick (Qiagen).

The materials and methods used in Example 1 for RNA extraction and analysis are used to compare the methods of exosome isolation and/or purification.

TABLE 1
Total Number of Input Reads

| | | | HCC | |
|---|---|---|---|---|
| | PrCA smRC | | smRC biomarker | |
| | characterization | | discovery cohort | |
| Read mapping status | cohor t(reads) | | (reads) | |
| Total (post cutadapt) | 494 828 430 | | 409 592 240 | |
| Unmapped | 213 988 996 | (43.2%) | 138 477 962 | (33.8%) |
| Unmapped because | 10 370 278 | (2.1%) | 11 205 827 | (2.7%) |
| m >50 | | | | |
| Unmapped because | 38 714 298 | (7.8%) | 7 066 592 | (1.7%) |
| guidance failed | 86 369 038 | (17.5%) | 203 895 810 | (49.8%) |
| Uniquely mapped (U) | | | | |
| Multiply mapped | 2 136 572 | (0.4%) | 2 072 756 | (0.4%) |
| (m <3, R) | | | | |
| Multiply mapped with | 143 249 248 | (28.9%) | 46 873 293 | (11.4%) |
| u-rescue (P) | 229 618 286 | (46.4%) | 250 769 103 | (61.2%) |
| Primary alignments | | | | |
| (U + R + P) | | | | |

TABLE 2
RT-qPCR Assay Sequences of 3smRC Signature and Genomic Location

| | | | Major | | | |
|---|---|---|---|---|---|---|
| | | Region | Peak | Average | | |
| | Genomic | Length | length | Expression | | Adj.P |
| smRC | Location (hg38) | (bp) | (bp) | (log2cpm) | logFC | value |
| smRC_119591 | chr8: 137627017- | 166 | 15 | 2.25985 | 3.25754 | 0.01583 |
| | 137627182 | | | | | |
| smRC_125851 | chr9: 95513777- | 2504 | 15 | 1.08142 | 3.55365 | 0.00951 |
| | 95515830 | | | | | |
| smRC 135709 | chr10: 70817194- | 894 | 15 | 2.32754 | 3.49786 | 0.00418 |
| | 70818087 | | | | | |
| smRC_48615 | chr3: 103950043- | 3585 | 15 | 3.10244 | 2.51252 | 0.03956 |
| | 103953627 | | | | | |

| | | | Included |
|---|---|---|---|
| | | Target Assay for | in 3-smRC |
| smRC | Peak Consensus Sequence | RT-qPCR assay | signature |
| smRC_119591 | CCUCUUCUUAACACC | UUGUCCUCUUCUUAACACC | Yes |
| | (SEQ ID NO: 1) | (SEQ ID NO: 2) | |
| smRC_125851 | CCCCUUAUUUACCCC | UUUCCUCCCCUUAUUUACCCC | No |
| | (SEQ ID NO. 7) | (SEQ ID NO: 8) | |
| smRC 135709 | CCUUCCCGUACUACC | CUCCCUUCCCGUACUACC | Yes |
| | (SEQ ID NO: 3) | (SEQ ID NO: 4) | |
| smRC_48615 | CUCUUUACAGUGACC | UGUCUCUUUACAGUGACC | Yes |
| | (SEQ ID NO: 5) | (SEQ ID NO: 6) | |

TABLE 3
Overlap of smRCs to other RNA

| RNA | Bio- | | | Alternative |
|---|---|---|---|---|
| biotype | fluid | statistic | P-value | hypothesis |
| mRNA | cell | 0.527 | 0 | CDF x above CDF y |
| mRNA | exRNA | 0.106 | 1.32e−05 | CDF x above CDF y |
| lincRNA | cell | 0.00 | 1 | CDF x above CDF y |
| lincRNA | exRNA | 0.250 | 0 | CDF x above CDF y |
| miRNA | cell | 0.913 | 0 | CDF x above CDF y |
| miRNA | exRNA | 0.393 | 0 | CDF x above CDF y |
| snoRNA | cell | 1.000 | 0 | CDF x above CDF y |
| snoRNA | exRNA | 0.158 | 0 | CDF x above CDF y |
| mRNA | cell | 0.008 | 1 | CDF x below CDF y |
| mRNA | exRNA | 0.068 | .0098135 | CDF x below CDF y |
| lincRNA | cell | 0.689 | 0 | CDF x below CDF y |
| lincRNA | exRNA | 0.058 | 0.03455966 | CDF x below CDF y |
| miRNA | cell | 0 | 1 | CDF x below CDF y |
| miRNA | exRNA | 0.068 | 0.0098135 | CDF x below CDF y |
| snoRNA | cell | 0 | 1 | CDF x below CDF y |
| snoRNA | exRNA | 0.245 | 0 | CDF x below CDF y |

TABLE 4
Clinical Characteristics of
HCC Biomarker Discovery Cohort

| | | CLD, risk | |
|---|---|---|---|
| | HCC | for HCC | P- |
| | (n = 10) | n = 5) | Value |
| Age (Years) | 67 | 63 | 0.94 |
| Sex (Male) | 7 (70%) | 3 (60%) | 1 |
| Cirrhosis (Yes) | 8 (80%) | 4 (80%) | 1 |
| Etiology | | | |
| HCV | 4 (40%) | 2 (40%) | 1 |
| HBV | 3 (30%) | 1 (20%) | 1 |
| NASH | 3 (30%) | 2 (20%) | 1 |
| Tumor stage (BCLC) | | | |
| Early Stage (Stage A) | 6 (60%) | n.a. | n.a. |
| Intermediate Stage (BCLC B) | 2 (20%) | n.a. | n.a. |
| Advanced Stage (BCLC C) | 2 (20%) | n.a. | n.a. |
| Largest nodule (cm) | 3.5 | n.a. | n.a. |
| AFP (ng/mL*) | 20.6 | 4.6 | 0.46 |

Continuous variables are dispayed as median.
*Upper limit of normal 9 ng/mL.
AFP, alpha fetoprotein,
BCLC, Barcelona Clinic for LiverCancer,
HBV/HCV, chronic hepatitis B/C,
NASH, non-alcoholic steatohepatitis

TABLE 5
Clinical Characteristics of the HCC Biomarker Validation Cohort

| | Early | | CLD, risk | | | Healthy |
|---|---|---|---|---|---|---|
| | Stage HCC¹ | | for HCC¹ | | p- | liver |
| | (n = 105) | | (n = 85) | | Value² | (n = 19) |
| Age (years) | 66 | (58, 71) | 62 | (56, 67) | 0.009 | 52 |
| Sex (male) | 82 | (80%) | 47 | (55%) | <0.001 | 6 (32%) |
| Cirrhosis (Yes) | 68 | (67%) | 61 | (72%) | | 0 |
| Bilirubin (mg/dL) | 0.7 | | 1.0 | | 0.6 | 0.6 |
| Albumin (g/dL) | 3.7 | | 3.9 | | 0.018 | 3.7 |
| Platelets | 152.5 | | 122.5 | | 0.002 | 243 |
| (count/mm³) | | | | | | |
| Etiology | | | | | 0.003 | |
| HCV | 44 | (43%) | 22 | (41%) | 0.10 | n.a. |
| HBV | 18 | (18%) | 15 | (28%) | | n.a. |
| Alcohol | 9 | (8.8%) | 12 | (22%) | | n.a. |
| NASH | 13 | (13%) | 2 | (3.7%) | | n.a. |
| Other | 18 | (18%) | 3 | (6%) | | n.a. |
| Tumor stage | | | | | n.a | |
| (BCLC) | | | | | | |

| | | | | |
|---|---|---|---|---|
| Very Early (Stage 0) | 22 | (21%) | 0 | n.a. |
| Early (Stage A) | 83 | (79%) | 0 | n.a. |
| Single nodule | 92 | (90%) | 10 | n.a. |

| | | | | |
|---|---|---|---|---|
| Largest nodule (cm) | 2.9 | n.a. | | n.a. |
| AFP (ng/mL*) | 8 | 4 | <0.001 | ** |

1 Statistics presented: median (IQR); n (%);
2 Statistical tests performed: Wilcoxon rank-sum test; chi-squaretest of independence;
*Upper limit of normal 9 ng/mL.
AFP, alpha fetoprotein,
BCLC, Barcelona Clinic for Liver Cancer,
HBV/HCV, chronic hepatitis B/C,
NASH, non-alcoholic steatohepatitis,
n.a., not applicable.

### REFERENCES

Chen, et al. Exosomal PD-L1 contributes to immunosuppression and is associated with anti-PD-1 response. Nature 560, 382 (2018).
European Association for the Study of the Liver, EASL Clinical Practice Guidelines: Management of hepatocellular carcinoma. J. Hepatol. 69(1), 182-236 (2018).
Felden, et al. Liquid biopsy in the clinical management of hepatocellular carcinoma. Gut 69, 2025-30 (2020).
Ghosh et al. Rapid Isolation of Extracellular Vesicels from Cell Culture and Biological Fluids using a Synthetic Peptide with Specific Affinity for Heat Shock Proteins. PlosOne 9(10), e110443 (2014)
Hoshino, et al. Tumour exosome integrins determine organotropic metastasis. Nature 527, 329-335 (2015).
Jemal, et al. Annual Report to the Nation on the Status of Cancer, 1975-2014, Featuring Survival. J. Natl. Cancer Inst. 109(9), 1-22 (2017).
Jeppesen, et al. Reassessment of Exosome Composition. Cell 177, 428-445.e18 (2019).
Jin, et al. Evaluation of Tumor-Derived Exosomal miRNA as Potential Diagnostic Biomarkers for Early-Stage Non-Small Cell Lung Cancer Using Next-Generation Sequencing. Clin. Cancer Res. 23, 5311-5319 (2017).
Kim, et al. Broken flow symmetry explains the dynamics of small particles in deterministic lateral displacement arrays. Proc Natl Acad Sci USA 114, E5034-E5041 (2017).
Kosaka, et al. Versatile roles of extracellular vesicles in cancer. J. Clin. Invest. 126, 1163-1172 (2016).
Kowal, et al. Proteomic comparison defines novel markers to characterize heterogeneous populations of extracellular vesicle subtypes. Proc. Natl. Acad. Sci. U.S.A 113, E968-77 (2016).
Lee, et al. Circulating exosomal noncoding RNAs as prognostic biomarkers in human hepatocellular carcinoma. Int J Cancer 144, 444-1452 (2019).
Llovet et al. Hepatocellular carcinoma. Nature Reviews Disease Primers 2, 16018-23 (2018)
Law, et al. voom: Precision weights unlock linear model analysis tools for RNA-seq read counts. Genome Biol 15, R29 (2014).
Martin Cutadapt removes adapter sequences from high-throughput sequencing reads. EMBnet.journal, 17(1),10-2 (2011).
Marrero, et al. Diagnosis, Staging, and Management of Hepatocellular Carcinoma: 2018 Practice Guidance by the American Association for the Study of Liver Diseases. Hepatology 68, 723-750 (2018).
Mathieu, et al. Specificities of secretion and uptake of exosomes and other extracellular vesicles for cell-to-cell communication. Nat. Cell Biol. 21, 9-17 (2019).
Mjelle et al. Comprehensive transcriptomic analyses of tissue, serum, and serum exosomes from hepatocellular carcinoma patients. BMC Cancer 19, 1007 (2019).
Murillo, et al. exRNA Atlas Analysis Reveals Distinct Extracellular RNA Cargo Types and Their Carriers Present across Human Biofluids. Cell 177, 463-477.e15 (2019).
Onuchic, et al. Epigenomic Deconvolution of Breast Tumors Reveals Metabolic Coupling between Constituent Cell Types. Cell Rep 17, 2075-2086 (2016).
Oussalah, et al. Plasma mSEPT9: A Novel Circulating Cell-free DNA-Based Epigenetic Biomarker to Diagnose Hepatocellular Carcinoma. EBioMedicine 30, 138-147 (2018).
Pepe, et al. Phases of biomarker development for early detection of cancer. J. Natl. Cancer Inst. 93, 1054-1061 (2001).
Pepe, The Statistical Evaluation of Medical Tests for Classification and Prediction. (Oxford University Press, 2003).
Qu, et al. Detection of early-stage hepatocellular carcinoma in asymptomatic HBsAg-seropositive individuals by liquid biopsy. Proc Natl Acad Sci USA 16, 6308-6312 (2019).
Rozowsky, et al. exceRpt: A Comprehensive Analytic Platform for Extracellular RNA Profiling. Cell Syst 8, 352-357.e3 (2019).
Singal, et al. Utilization of hepatocellular carcinoma surveillance among American patients: a systematic review. J. Gen. Intern. Med. 27, 861-867 (2012).
Skog, et al. Glioblastoma microvesicles transport RNA and proteins that promote tumour growth and provide diagnostic biomarkers. Nat. Cell Biol. 10, 1470-1476 (2008).
Smith, et al. Correcting for optimistic prediction in small data sets. Am J Epidemiol 180, 318-324 (2014).
Sun, et al. Purification of HCC-specific extracellular vesicles on nanosubstrates for early HCC detection by digital scoring. Nat Commun 11, 4489 (2020).
Sun, et al. Emerging role of exosome-derived long non-coding RNAs in tumor microenvironment. Mol. Cancer 17, 82 (2018).
Thery, et al. Minimal information for studies of extracellular vesicles 2018 (MISEV2018): a position statement of the International Society for Extracellular Vesicles and update of the MISEV2014 guidelines. J Extracell Vesicles 7, 1535750 (2018).
Tzartzeva, et al. Surveillance Imaging and Alpha Fetoprotein for Early Detection of Hepatocellular Carcinoma in Patients With Cirrhosis: A Meta-analysis. Gastroenterology 154, 1706-1718.el (2018).
van Niel, et al. Shedding light on the cell biology of extracellular vesicles. Nat. Rev. Mol. Cell Biol. 19, 213-228 (2018).
Villanueva. Hepatocellular Carcinoma. N. Engl. J. Med. 380, 1450-1462 (2019).
Wunsch, et al. Nanoscale lateral displacement arrays for the separation of exosomes and colloids down to 20 nm. Nat. Nanotechnol. 11, 936-940 (2016).
Xu, et al. Circulating tumour DNA methylation markers for diagnosis and prognosis of hepatocellular carcinoma. Nat Mater 16, 1155-1161 (2017).
Yang, et al. Multiparametric plasma EV profiling facilitates diagnosis of pancreatic malignancy. Sci. Transl. Med. 9, (2017).
Zhang, et al. Microenvironment-induced PTEN loss by exosomal microRNA primes brain metastasis outgrowth. Nature 527, 100-104 (2015).
Zhang, et al. Multiple distinct small RNAs originate from the same microRNA precursors. Genome Biol 11(8):R81 (2010).

1. A method of detecting three, small unannotated non-coding RNAs specific for hepatocellular carcinoma in a subject, comprising:
a. isolating exosomes from a sample from the subject;
b. extracting RNA from the exosomes;
c. contacting RNA from the exosomes with at least one primer which is a synthetic nucleic acid and wherein the primer sequence comprises the nucleotide sequence of SEQ ID NOs: 1 or 2 or a fragment or variant thereof or the nucleotide sequence complementary to SEQ ID NOs: 1 or 2 or a fragment or variant thereof;
d. further contacting RNA from the exosomes with at least one primer which is a synthetic nucleic acid and wherein the primer sequence comprises the nucleotide sequence of SEQ ID NOs: 3 or 4 or a fragment or variant thereof or the nucleotide sequence complementary to SEQ ID NOs: 3 or 4 or a fragment or variant thereof;
e. further contacting RNA from the exosomes with at least one primer which is a synthetic nucleic acid and wherein the primer sequence comprises the nucleotide sequence of SEQ ID NOs: 5 or 6 or a fragment or variant thereof or the nucleotide sequence complementary to SEQ ID NOs: 5 or 6 or a fragment or variant thereof;
f. subjecting the RNA and the primers to amplification conditions; and
g. determining the presence of amplification products, wherein the presence of amplification products indicates the presence of small unannotated non-coding RNAs specific for hepatocellular carcinoma in the sample.

2. The method of claim 1, wherein the subject is at risk for hepatocellular carcinoma.

3. The method of claim 1, wherein the subject is suffering from cirrhosis of the liver, hepatitis C virus infection, hepatitis B virus infection, non-alcoholic fatty liver disease or combinations thereof.

4. The method of claim 1, wherein the sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

5. The method of claim 1, wherein the sample is selected from blood, serum, plasma, and urine.

6. The method of claim 1, wherein the exosomes are purified from the sample by a method selected from the group selected from ultracentrifugation, use of VN96 peptide, and use of a polymer which precipitates the exosome from the sample.

7. A synthetic nucleic acid comprising at least about 10 nucleotides of the isolated nucleic acid comprising the nucleic acid sequence of any of SEQ ID NOs: 1-6 or fragments or variants thereof.

8. A synthetic nucleic acid comprising at least about 10 nucleotides complementary to the isolated nucleic acid having the nucleic acid sequence of any of SEQ ID NOs: 1-6 or fragments or variants thereof.

9. A method for detecting and/or diagnosing hepatocellular carcinoma in a subject, comprising:
a. isolating exosomes from a sample from the subject;
b. extracting RNA from the exosomes;
c. assaying the RNA extracted from the exosomes for the levels of three, small unannotated non-coding RNAs, wherein the three, small unannotated non-coding RNAs comprise the nucleotide sequences SEQ ID NOs: 1, 3 and 5;
d. comparing levels of the three, small unannotated non-coding RNAs from the sample to reference levels of the three, small unannotated non-coding RNAs; and
e. detecting and/or diagnosing that the subject has hepatocellular carcinoma when the levels of the three, small unannotated non-coding RNAs are increased compared to the reference levels.

10. The method of claim 9, wherein the subject is at risk for hepatocellular carcinoma.

11. The method of claim 9, wherein the subject is suffering from cirrhosis of the liver, hepatitis C virus infection, hepatitis B virus infection, non-alcoholic fatty liver disease or combinations thereof.

12. The method of claim 9, wherein the sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

13. The method of claim 9, wherein the sample is selected from blood, serum, plasma, and urine.

14. The method of claim 9, wherein the exosomes are isolated from the sample by a method selected from the group selected from ultracentrifugation, use of VN96 peptide, and use of a polymer which precipitates the exosome from the sample.

15. The method of claim 9, wherein the level of the three, small unannotated non-coding RNAs is detected by polymerase chain reaction.

16. The method of claim 15, wherein the polymerase chain reaction is performed using primers comprising a nucleotide sequence of SEQ ID NOs: 1-6 or a fragment or variant thereof or a nucleotide sequence complementary to SEQ ID NOs: 1-6 or a fragment or variant thereof.

17. The method of claim 9, wherein the reference level of the three, small unannotated non-coding RNAs is from a subject not suffering from hepatocellular carcinoma and has an elevated risk for hepatocellular carcinoma.

18. A method for detecting and/or diagnosing hepatocellular carcinoma in a subject, comprising:
a. isolating exosomes from a first sample from the subject;
b. extracting RNA from the exosomes;
c. assaying the RNA extracted from the exosomes for the levels of three, small unannotated non-coding RNAs, wherein the three, small unannotated non-coding RNAs comprise the nucleotide sequences SEQ ID NOs: 1, 3, and 5;
d. comparing the levels of the three, small unannotated non-coding RNAs from the sample to reference levels of the three, small unannotated non-coding RNAs;
e. assaying the level of alpha-fetoprotein in a second sample from the subject;
f. comparing the level of alpha-fetoprotein in the second sample from the subject to a reference level of alpha-fetoprotein; and
g. detecting and/or diagnosing that the subject has hepatocellular carcinoma when the levels of the three, small unannotated non-coding RNAs and the alpha-fetoprotein are increased compared to the reference levels.

19. The method of claim 18, wherein the subject is at risk for hepatocellular carcinoma.

20. The method of claim 18, wherein the subject is suffering from cirrhosis of the liver, hepatitis C virus infection, hepatitis B virus infection, non-alcoholic fatty liver disease or combinations thereof.

21. The method of claim 18, wherein the first sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

22. The method of claim 18, wherein the first sample is selected from blood, serum, plasma, and urine.

23. The method of claim 18, wherein the exosomes are isolated from the first sample by a method selected from the group selected from ultracentrifugation, use of VN96 peptide, and use of a polymer which precipitates the exosome from the sample.

24. The method of claim 18, wherein the level of the three, small unannotated non-coding RNAs is detected by polymerase chain reaction.

25. The method of claim 24, wherein the polymerase chain reaction is performed using primers comprising a nucleotide sequence of SEQ ID NOs: 1-6 or a fragment or variant thereof or a nucleotide sequence complementary to SEQ ID NOs: 1-6 or a fragment or variant thereof.

26. The method of claim 18, wherein the reference level of the three, small unannotated non-coding RNAs is from a subject not suffering from hepatocellular carcinoma and has an elevated risk for hepatocellular carcinoma.

27. The method of claim 18, wherein the second sample is selected from the group consisting of blood, serum, plasma, and urine.

28. The method of claim 18, wherein the level of alpha-fetoprotein is detected using a method selected from the group consisting of flow cytometry, quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

29. The method of claim 18, wherein the reference level of the alpha-fetoprotein is from a subject not suffering from hepatocellular carcinoma or liver disease.

30. The method of claim 18, wherein the first and second sample are the same sample from the subject.

31. The method of claim 18, wherein the first and the second sample are different samples from the subject.

32. A method of detecting and/or diagnosing hepatocellular carcinoma in a subject comprising:
a. isolating exosomes from a sample from the subject;
b. extracting RNA from the exosomes;
c. assaying the RNA extracted from the exosomes for the levels of three, small unannotated non-coding RNAs, wherein the three, small unannotated non-coding RNAs comprise the nucleotide sequences SEQ ID NOs: 1-3 and are denoted smRC 119591, smRC 135709 and smRC 48615;
d. calculating the risk of hepatocellular carcinoma using the levels of the small unannotated non-coding RNAs in the formula:
HCC probability˜(1+exp(−coef))\hat{ }(−1), wherein
coef=epsilon+alpha*smrc_119591+beta*smrc_135709+gamma*smrc_48615
and wherein alpha=[1.5, 1.9]; beta=[1.5, 1.9]; gamma=[5, 1.2]; and epsilon=[1.2, 0.8]; and
e. detecting and/or diagnosing the subject has hepatocellular carcinoma when a subject has an overexpression of the three, small unannotated non-coding RNAs according to the formula.

33. The method of claim 32, wherein detecting and/or diagnosing that the subject has hepatocellular carcinoma includes comparing the HCC probability to a threshold value and wherein when the HCC probability exceeds the threshold value, automatically detecting and/or diagnosing the patient as having hepatocellular carcinoma.

34. The method of claim 33, wherein the threshold value is greater than or equal to 40%.

35. The method of claim 33, wherein the threshold value is greater than or equal to 60%.

36. The method of claim 32, wherein the subject is at risk for hepatocellular carcinoma.

37. The method of claim 32, wherein the subject is suffering from cirrhosis of the liver, hepatitis C virus infection, hepatitis B virus infection, non-alcoholic fatty liver disease or combinations thereof.

38. The method of claim 32, wherein the sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

39. The method of claim 32, wherein the sample is selected from blood, serum, plasma, and urine.

40. The method of claim 32, wherein the exosomes are isolated from the sample by a method selected from the group selected from ultracentrifugation, use of VN96 peptide, and use of a polymer which precipitates the exosome from the sample.

41. The method of claim 32, wherein the level of the three, small unannotated non-coding RNAs is detected by polymerase chain reaction.

42. The method of claim 41, wherein the polymerase chain reaction is performed using primers comprising a nucleotide sequence of SEQ ID NOs: 1-6 or a fragment or variant thereof or a nucleotide sequence complementary to SEQ ID NOs: 1-6 or a fragment or variant thereof.

43. The method of claim 32, further comprising the steps of assaying the level of alpha-fetoprotein in a sample from the subject and comparing the level of alpha-fetoprotein in the sample from the subject to a reference level of alpha-fetoprotein.

44. A method for treating hepatocellular carcinoma in a subject, comprising:
a. isolating exosomes from a sample from the subject;
b. extracting RNA from the exosomes;
c. assaying the RNA extracted from the exosomes for three, small unannotated non-coding RNAs, wherein the three, small unannotated non-coding RNAs comprise the nucleotide sequences SEQ ID NOs: 1-3;
d. comparing the levels of the three, small unannotated non-coding RNAs from the sample to reference levels of the three, small unannotated non-coding RNAs;
e. detecting and/or diagnosing that the subject has hepatocellular carcinoma when the levels of the three, small unannotated non-coding RNAs are increased compared to the reference levels; and
f. treating the subject, wherein the treatment is selected from the group consisting of surgical therapies, tumor ablation and immune based therapies.

45. The method of claim 44, wherein the subject is at risk for hepatocellular carcinoma.

46. The method of claim 44, wherein the subject is suffering from cirrhosis of the liver, hepatitis C virus infection, hepatitis B virus infection, non-alcoholic fatty liver disease or combinations thereof.

47. The method of claim 44, wherein the sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

48. The method of claim 44, wherein the sample is selected from blood, serum, plasma, and urine.

49. The method of claim 44, wherein the exosomes are isolated from the sample by a method selected from the group selected from ultracentrifugation, use of VN96 peptide, and use of a polymer which precipitates the exosome from the sample.

50. The method of claim 44, wherein the level of the three, small unannotated non-coding RNAs is detected by polymerase chain reaction.

51. The method of claim 50, wherein the polymerase chain reaction is performed using primers comprising a nucleotide sequence of SEQ ID NOs: 1-6 or a fragment or variant thereof or a nucleotide sequence complementary to SEQ ID NOs: 1-6 or a fragment or variant thereof.

52. The method of claim 44, wherein the reference level of the three, small unannotated non-coding RNAs is from a subject not suffering from hepatocellular carcinoma and has an elevated risk for hepatocellular carcinoma.

53. The method of claim 44, further comprising assaying the level of alpha-fetoprotein in a sample from the subject; comparing the level of alpha-fetoprotein in the sample from the subject to a reference level of alpha-fetoprotein; and detecting that the subject has hepatocellular carcinoma when the levels of the three small unannotated non-coding RNAs and the alpha-fetoprotein are increased compared to the levels in the healthy control.

54. The method of claim 44, wherein the surgical therapies are selected from the group consisting of resection and liver transplantation.

55. The method of claim 44, further comprising confirming the detection and/or diagnosis of hepatocellular carcinoma using methods selected from the group consisting of ultrasound, detection of alpha-fetoprotein, magnetic resonance imaging (MRI), computed tomography (CT), biopsy, and combinations thereof.

56. A method of treating hepatocellular carcinoma in a subject comprising:
a. isolating exosomes from a sample from the subject;
b. extracting RNA from the exosomes;
c. assaying the RNA extracted from the exosomes for the levels of three small unannotated non-coding RNAs, wherein the three, small unannotated non-coding RNAs comprise the nucleotide sequences SEQ ID NOs: 1, 3, and 5 and are denoted smRC 119591, smRC 135709 and smRC 48615;
d. calculating the risk of hepatocellular carcinoma using the levels of the small unannotated non-coding RNAs in the formula
HCC probability˜(1+exp(−coef))\hat{ }(−1), wherein,
coef=epsilon+alpha*smrc_119591+beta*smrc_135709+gamma*smrc_48615
and wherein, alpha=[1.5, 1.9]; −beta=[1.5, 1.9]; gamma=[5, 1.2]; and epsilon=[1.2, 0.8];
e. detecting and/or diagnosing the subject has hepatocellular carcinoma when a subject has an overexpression of the three, small unannotated non-coding RNAs according to the formula; and
f. treating the subject, wherein the treatment is selected from the group consisting of surgical therapies and tumor ablation and immune based therapies.

57. The method of claim 56, wherein detecting and/or diagnosing that the subject has hepatocellular carcinoma includes comparing the HCC probability to a threshold value and wherein when the HCC probability exceeds the threshold value, automatically detecting and/or diagnosing the patient as having hepatocellular carcinoma.

58. The method of claim 57, wherein the threshold value is greater than or equal to 40%.

59. The method of claim 57, wherein the threshold value is greater than or equal to 60%.

60. The method of claim 56, wherein the subject is at risk for hepatocellular carcinoma.

61. The method of claim 56, wherein the subject is suffering from cirrhosis of the liver, hepatitis C virus infection, hepatitis B virus infection, non-alcoholic fatty liver disease or combinations thereof.

62. The method of claim 56, wherein the sample is selected from the group consisting of blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes.

63. The method of claim 56, wherein the sample is selected from blood, serum, plasma, and urine.

64. The method of claim 56, wherein the exosomes are isolated from the sample by a method selected from the group selected from ultracentrifugation, use of VN96 peptide, and use of a polymer which precipitates the exosome from the sample.

65. The method of claim 56, wherein the level of the three, small unannotated non-coding RNAs is detected by polymerase chain reaction.

66. The method of claim 65, wherein the polymerase chain reaction is performed using primers comprising a nucleotide sequence of SEQ ID NOs: 1-6 or a fragment or variant thereof or a nucleotide sequence complementary to SEQ ID NOs: 1-6 or a fragment or variant thereof.

67. The method of claim 56, further comprising the steps of assaying the level of alpha-fetoprotein in a sample from the subject and comparing the level of alpha-fetoprotein in a sample from the subject to a reference level of alpha-fetoprotein.

68. The method of claim 56, wherein the surgical therapies are selected from the group consisting of resection and liver transplantation.

69. The method of claim 56, further comprising confirming the detection and/or diagnosis of hepatocellular carcinoma using methods selected from the group consisting of ultrasound, the detection of alpha-fetoprotein, magnetic resonance imaging (MRI), computed tomography (CT), biopsy and combinations thereof.

70. A kit for practicing the methods of any of claims 1, 9, 32, 44, and 56 comprising primers and/or probes having a nucleotide sequence of SEQ ID NOs: 1-6 or a fragment or variant thereof or a nucleotide sequence complementary to SEQ ID NOs: 1-6 or a fragment or variant thereof, reagents for isolating and/or purifying exosomes from a sample, reagents for extracting RNA from the exosomes, additional reagents for detecting the three small unannotated non-coding RNAs, reference levels or the means for obtaining reference levels for the three small unannotated non-coding RNAs, and instructions for use.

71. A kit for practicing the method of claims 18, 43, 53, and 67, comprising primers and/or probes having a nucleotide sequence of SEQ ID NOs: 1-6 or a fragment or variant thereof or a nucleotide sequence complementary to SEQ ID NOs: 1-6 or a fragment or variant thereof, reagents for isolating and/or purifying exosomes from a sample, reagents for extracting RNA from the exosomes, additional reagents for detecting the three small unannotated non-coding RNAs, reference levels or the means for obtaining reference levels for the three small unannotated non-coding RNAs, reagents for purifying AFP from a sample, reagents for detecting AFP in a sample, reference levels or means for obtaining reference levels in a control sample for AFP, and instructions for use.


SEQUENCE LISTING (9 sequences)

SEQ ID NO: 1 - RNA, 15 nt, Homo sapiens
ccucuucuuaacacc

SEQ ID NO: 2 - RNA, 19 nt, Homo sapiens
uuguccucuucuuaacacc

SEQ ID NO: 3 - RNA, 15 nt, Homo sapiens
ccuucccguacuacc

SEQ ID NO: 4 - RNA, 18 nt, Homo sapiens
cucccuucccguacuacc

SEQ ID NO: 5 - RNA, 15 nt, Homo sapiens
cucuuuacagugacc

SEQ ID NO: 6 - RNA, 18 nt, Homo sapiens
ugucucuuuacagugacc

SEQ ID NO: 7 - RNA, 15 nt, Homo sapiens
ccccuuauuuacccc

SEQ ID NO: 8 - RNA, 21 nt, Homo sapiens
uuuccuccccuuauuuacccc

SEQ ID NO: 9 - RNA, 10 nt, Artificial Sequence
  synthetic adaptor
aaaaaaaaaa